(19) United States
(12) Patent Application Publication
(10) Pub. No.: US 2026/0034034 A1
(43) Pub. Date: Feb. 5, 2026

(54) COMPOSITE MICROCAPSULES

(71) Applicant: Firmenich SA, Satigny (CH)

(72) Inventors: Amal ELABBADI, Satigny (CH); Philipp ERNI, Satigny (CH); Lahoussine OUALI, Satigny (CH)

(21) Appl. No.: 18/993,185

(22) PCT Filed: Jul. 20, 2023
(86) PCT No.: PCT/EP2023/070184

§ 371 Date: Jan. 10, 2025

(30) Foreign Application Priority Data
Jul. 21, 2022 (EP) 22186312.9

(51) Int. Cl. A61K 8/11; A61Q 13/00; B01J 13/16; C11D 1/14; C11D 1/22; C11D 1/29; C11D 1/72; C11D 1/83; C11D 1/831; C11D 3/00; C11D 3/22; C11D 3/37; C11D 3/382; C11D 3/50; C11D 17/00

(52) U.S. Cl. CPC A61K 8/11; A61Q 13/00; B01J 13/16; C11D 1/83; C11D 1/831; C11D 3/001; C11D 3/222; C11D 3/3726; C11D 3/382; C11D 3/505; C11D 17/0039; C11D 1/146; C11D 1/22; C11D 1/29; C11D 1/72

(57) ABSTRACT

Disclosed herein are microcapsules including both a core and a composite shell including a plant-based coacervate and a polymeric material.

### CROSS-REFERENCE TO RELATED APPLICATIONS

This application is a U.S. National Phase Application of International Patent Application No. PCT/EP23/70184, filed Jul. 20, 2023, which claims priority European Patent Application No. 22186312.9, filed Jul. 21, 2022, the contents of which are hereby incorporated by reference herein.

### FIELD OF THE INVENTION

The invention relates to microcapsules comprising both a core and a composite shell comprising a plant-based coacervate and a polymeric material.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing active ingredients, for example a perfume, are needed to protect and later release the core payload when triggered. A key requirement from the industry regarding these systems is to survive suspension in challenging bases without physically dissociating or degrading. This is referred to as chemical stability for the delivery system. For instance, fragranced personal and household cleansers containing high levels of aggressive surfactant detergents are very challenging for the stability of microcapsules. High levels of surfactants also increase the speed of diffusion of actives out of the microcapsules. This leads to leakage of the actives during storage and a reduced impact when the microcapsules are triggered to release. In addition, the mechanical stability of microcapsules can be compromised by physical forces, such as crushing, or other methods that compromise the integrity of the microcapsules.

In addition to the performance in terms of stability and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

### SUMMARY OF THE INVENTION

It has now been found that performing microcapsules encapsulating a hydrophobic material such as perfume oil could be obtained by forming a homogeneous composite shell comprising (or made of) a plant-based coacervate material and a polymeric material.

A first object of the present invention is a core-shell microcapsule comprising:
a core comprising a hydrophobic material, preferably a perfume oil, and
a polymeric shell comprising:
   a polymeric material, and
   a coacervate comprising a first polyelectrolyte and a second polyelectrolyte, wherein the first polyelectrolyte comprises a plant-protein.

A second object of the invention is a process for preparing a core-shell microcapsule slurry comprising the steps of:
(i) providing a hydrophobic phase comprising a hydrophobic material and at least a polyfunctional monomer;
(ii) mixing a first polyelectrolyte and second polyelectrolyte in a dispersing phase under conditions sufficient to not form a suspension of complex coacervate;
(iii) adding the hydrophobic phase into the dispersing phase to form a two phases dispersion and applying sufficient condition to form a coacervate,
(iv) providing conditions sufficient to induce interfacial polymerization to form a core-shell microcapsule slurry.

A third object is a core-shell microcapsule slurry obtainable by the process defined above.

The present invention also relates to a consumer product in the form of a home care, fabric care or personal care products comprising said microcapsules or said microcapsule slurry.

### DETAILED DESCRIPTION OF THE INVENTION

Unless stated otherwise, percentages (%) are meant to designate a percentage by weight of a composition.

By “hydrophobic material”, it is meant any hydrophobic material—single material or a mixture of materials—which forms a two-phase dispersion when mixed with water.

By “ingredient”, it is meant a single compound or a combination of ingredients.

By “perfume or flavour oil”, it is meant a single perfuming or flavouring compound or a mixture of several perfuming or flavouring compounds.

By “consumer product” or “end-product” it is meant a manufactured product ready to be distributed, sold and used by a consumer.

A “microcapsule”, or the similar, in the present invention it is meant that core-shell microcapsules have a particle size distribution in the micron range (e.g. a mean diameter (Dv(50) comprised between about 1 and 3000 microns) and comprise an external polymeric-based shell and an internal hydrophobic phase enclosed by the external shell. According to an embodiment, microcapsules have a mean diameter comprised between 1 and 500 microns, preferably from 2 and 200 microns, more preferably between 4 and 100 microns, even more preferably between 4 and 50 microns.

According to the invention, the wordings “mean diameter” or “mean size” are used indifferently. According to an embodiment, microcapsules are not agglomerated. According to another embodiment, microcapsules are partly agglomerated. Still according to another embodiment, the totality of the microcapsules is agglomerated.

By “microcapsule slurry”, it is meant microcapsule(s) that is (are) dispersed in a liquid. According to an embodiment, the slurry is an aqueous slurry, i.e the microcapsule(s) is (are) dispersed in an aqueous phase.

The microcapsule of the present invention is a composite microcapsule. By “composite microcapsule”, it is meant a core-shell microcapsule having a composite shell, namely a shell comprising at least two different materials (a first plant-based coacervate material and a second polymeric material). According to the invention, “coacervate” or “hydrogel” can be used indifferently. By hydrogel, it is meant a polymer network swollen with water.

According to an embodiment, by composite shell, it should be also understood a homogeneous shell, meaning that the plant-based coacervate and the polymeric material are homogeneously dispersed within the shell. In other words, the microcapsule does not contain two distinctive layers but a single composite shell. The microcapsule according to the invention does not comprise an inner shell of a polymeric material and an outer shell of a coacervate.

According to a particular embodiment, the polymeric shell comprises the reaction product of the polymeric material with the coacervate.

### FIGURES

FIGS. 1 to 3 represent respectively optical microscopy and SEM images of the invention's microcapsules.

FIG. 4 and FIG. 5 represents respectively olfactive performance of microcapsules A and microcapsules B in a fabric softener and in a liquid detergent.

### MICROCAPSULES

A first object of the present invention is a core-shell microcapsule comprising:
a core comprising a hydrophobic material, preferably a perfume oil, and
a polymeric shell comprising:
   a polymeric material, and
   a coacervate comprising a first polyelectrolyte and a second polyelectrolyte, wherein the first polyelectrolyte comprises a plant-protein.
      Another object is a slurry comprising microcapsules as defined above.

### Hydrophobic Material

According to an embodiment, the core is an oil-based core.

The hydrophobic material according to the invention can be “inert” material like solvents or active ingredients.

When the hydrophobic material is an active ingredient, it is preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to an embodiment, the hydrophobic material comprises a phase change material (PCM).

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, modulators, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils (for example Thyme oil), and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0^{2,7}]undecan-4-one, 1-methoxy-3-hexanethiol, 2-ethyl-4,4-dimethyl-1,3-oxathiane, 2,2,7/8,9/10-tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 3-(3,3/1,1-dimethyl-5-indanyl)propanal, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{(1S)-1-[(1R)-3,3-dimethylcyclohexyl]ethoxy}-2-oxoethyl propionate, 3-methyl-5-cyclopentadecen-1-one, 4,6,6,7,8,8-hexamethyl-1,3,4,6,7,8-hexahydrocyclopenta[g]isochromene, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonane and/or 3-(3-isopropyl-1-phenyl)butanal.
   It is also understood that said ingredients may also be compounds known to release in a controlled manner various types of perfuming compounds also known as proper fume or profragrance. Non-limiting examples of suitable proper fumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, triethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

Preferred perfuming ingredients are those having a high steric hindrance (bulky materials) and in particular those from one of the following groups:
Group 1: perfuming ingredients comprising a cyclohexane, cyclohexene, cyclohexanone or cyclohexenone ring substituted with at least one linear or branched C₁ to C₄ alkyl or alkenyl substituent;
Group 2: perfuming ingredients comprising a cyclopentane, cyclopentene, cyclopentanone or cyclopentenone ring substituted with at least one linear or branched C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one linear or branched C₅ to C₈ alkyl or alkenyl substituent or with at least one phenyl substituent and optionally one or more linear or branched C₁ to C₃ alkyl or alkenyl substituents;
Group 4: perfuming ingredients comprising at least two fused or linked C₅ and/or C₆ rings;
Group 5: perfuming ingredients comprising a camphor-like ring structure;
Group 6: perfuming ingredients comprising at least one C₇ to C₂₀ ring structure;
Group 7: perfuming ingredients having a log P value above 3.5 and comprising at least one tert-butyl or at least one trichloromethyl substitutent;

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3RS,3aRS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), (1′R)-2-[2-(4′-methyl-3′-cyclohexen-1′-yl)propyl]cyclopentanone, alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0^{2,6}]dec-3-en-8-yl 2-methylpropanoate and (1RS,2SR,6RS,7RS,8SR)-tricyclo[5.2.1.0^{2,6}]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0^{2,7}]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), 9-hexadecen-16-olide (origin: Firmenich SA, Geneva, Switzerland), pentadecenolide (origin: Firmenich SA, Geneva, Switzerland), 3-methyl-(4/5)-cyclopentadecenone, (origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

According to a particular embodiment, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt %, preferably 25-98% of a perfume oil comprising at least 15 wt % of high impact perfume raw materials having a Log T<−4, and
0-75 wt %, preferably 2-75% of a density balancing material having a density greater than 1.07 g/cm³.
   “High impact perfume raw materials” should be understood as perfume raw materials having a Log T<−4. The odor threshold concentration of a chemical compound is determined in part by its shape, polarity, partial charges and molecular mass. For convenience, the odor threshold concentration is presented as the common logarithm of the threshold concentration, i.e., Log[Threshold] (“Log T”).
   A “density balancing material” should be understood as a material having a density greater than 1.07 g/cm³ and having preferably low or no odor.
   The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.
   The nature of high impact perfume raw materials having a Log T<−4 and density balancing material having a density greater than 1.07 g/cm³ are described in WO2018115250, the content of which are included by reference.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl)ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3(4H)-one, (2E,6Z)-2,6-nonadien-1-ol, (4Z)-4-dodecenal, (+−)-4-hydroxy-2,5-dimethyl-3(2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl)phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one(, 2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2(3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl)ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl)pent-1-en-3-one, indol, 7-propyl-2H,4H-1,5-benzodioxepin-3-one, ethyl praline, (4-methylphenoxy)acetaldehyde, ethyl tricyclo[5.2.1.0.^{2,6}]decane-2-carboxylate, (+)-(1′S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxydes, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70% by weight based on the total weight of the perfume raw materials having a Log T<−4.

According to an embodiment, perfume raw materials having a Log T<−4 comprise between 20 and 70% by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

According to an embodiment, the oil-based core (or perfume formulation) comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of Bulky materials of groups 1 to 6, preferably 3 to 6 as defined previously and
   at least 15%, preferably at least 20%, more preferably at least 25%, even more preferably at least 30% of high impact perfume materials having a Log T<−4 as defined previously,
      optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜ−δD_{fragrance})²+(δPₛₒₗᵥₑₙₜ−δP_{fragrance})²+(δHₛₒₗᵥₑₙₜ−δH_{fragrance})²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

In a particular embodiment, at least 90% of the perfume oil, preferably at least 95% of the perfume oil, most preferably at least of 98% of the perfume oil has at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11.

According to an embodiment, the perfuming formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a c log P of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably, as examples the following ingredients can be listed as modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

According to a particular embodiment, the hydrophobic material is free of any active ingredient (such as perfume). According to this particular embodiment, it comprises, preferably consists of hydrophobic solvents, preferably chosen in the group consisting of isopropyl myristate, triglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

By “flavor oil”, it is meant here a flavoring ingredient or a mixture of flavoring ingredients, solvents or adjuvants of current use for the preparation of a flavoring formulation, i.e. a particular mixture of ingredients which is intended to be added to an edible composition or chewable product to impart, improve or modify its organoleptic properties, in particular its flavor and/or taste. Flavoring ingredients are well known to a person skilled in the art and their nature does not warrant a detailed description here, which in any case would not be exhaustive, the skilled flavorist being able to select them on the basis of his general knowledge and according to the intended use or application and the organoleptic effect it is desired to achieve. Many of these flavoring ingredients are listed in reference texts such as in the book by S. Arctander, Perfume and Flavor Chemicals, 1969, Montclair, N.J., USA, or its more recent versions, or in other works of similar nature such as Fenaroli's Handbook of Flavor Ingredients, 1975, CRC Press or Synthetic Food Adjuncts, 1947, by M. B. Jacobs, van Nostrand Co., Inc. Solvents and adjuvants of current use for the preparation of a flavoring formulation are also well known in the art.

In a particular embodiment, the flavor is a mint flavor. In a more particular embodiment, the mint is selected from the group consisting of peppermint and spearmint.

In a further embodiment, the flavor is a cooling agent or mixtures thereof.

In another embodiment, the flavor is a menthol flavor.

Flavors that are derived from or based on fruits where citric acid is the predominant, naturally-occurring acid include but are not limited to, for example, citrus fruits (e.g. lemon, lime), limonene, strawberry, orange, and pineapple. In one embodiment, the flavors food is lemon, lime or orange juice extracted directly from the fruit. Further embodiments of the flavor comprise the juice or liquid extracted from oranges, lemons, grapefruits, key limes, citrons, clementines, mandarins, tangerines, and any other citrus fruit, or variation or hybrid thereof. In a particular embodiment, the flavor comprises a liquid extracted or distilled from oranges, lemons, grapefruits, key limes, citrons, clementines, mandarins, tangerines, any other citrus fruit or variation or hybrid thereof, pomegranates, kiwifruits, watermelons, apples, bananas, blueberries, melons, ginger, bell peppers, cucumbers, passion fruits, mangos, pears, tomatoes, and strawberries.

In a particular embodiment, the flavor comprises a composition that comprises limonene, in a particular embodiment, the composition is a citrus that further comprises limonene.

In another particular embodiment, the flavor comprises a flavor selected from the group comprising strawberry, orange, lime, tropical, berry mix, and pineapple.

The phrase flavor includes not only flavors that impart or modify the smell of foods but include taste imparting or modifying ingredients. The latter do not necessarily have a taste or smell themselves but are capable of modifying the taste that other ingredients provides, for instance, salt enhancing ingredients, sweetness enhancing ingredients, umami enhancing ingredients, bitterness blocking ingredients and so on.

In a further embodiment, suitable sweetening components may be included in the particles described herein. In a particular embodiment, a sweetening component is selected from the group consisting of sugar (e.g., but not limited to sucrose), a Stevia component (such as but not limited to stevioside or rebaudioside A), sodium cyclamate, aspartame, sucralose, sodium saccharine, and Acesulfam K or mixtures thereof.

### Polymeric Material

According to the invention, the polymeric shell comprises a polymeric material.

According to an embodiment, the polymeric material is selected from the group consisting of polyurea, polyurethane, polyamide, polyester, polyacrylate, polysiloxane, polycarbonate, polysulfonamide, poly (beta aminoester), polylactic acid, poly (thiol-acrylate), polymers of urea and formaldehyde, melamine and formaldehyde, melamine and urea, or melamine and glyoxal and mixtures thereof.

According to a particular embodiment, the polymeric material is polyurea and/or polyurethane.

According to an embodiment, the polymeric material is a polyurea-based material and comprises the reaction product of a polyisocyanate with optionally an amine. According this particular embodiment, interfacial polymerization can be induced by addition of a polyamine reactant. Preferably, the reactant is selected from the group consisting of water-soluble guanidine salts and guanazole to form a polyurea material with the polyisocyanate. According to another embodiment, polyurea-based polymeric material is formed in absence of added polyamine reactant, and result only from the autopolymerization of the at least one polyisocyanate, preferably in the presence of a catalyst.

According to another embodiment, the polymeric material is a polyurethane-based material and comprises the reaction product of a polyisocyanate with a polyol. According to this particular embodiment, interfacial polymerization is induced by addition of a polyol reactant. Preferably the reactant is selected from the group consisting of monomeric and polymeric polyols with multiple hydroxyl groups available for reaction and mixtures thereof.

According to another embodiment, the polymeric material is a polyurea/polyurethane based material and comprises the reaction product of a polyisocyanate with a polyol and an amine. In that case interfacial polymerization is induced by addition of a mixture of the reactant mentioned under precedent first and second embodiments. Additionally, crosslinkers with both amino groups and hydroxyl groups can be used to generate polyurea/polyurethane materials. Furthermore, polyisocyanates with both urea and urethane functionalities can be used to generate polyurea/polyurethane materials.

According to another embodiment, the polymeric material is a polyamide-based material and comprises the reaction product of an acyl chloride with at least one amine, preferably at least two amines. According to a particular embodiment, the second polymeric material is a polyamide-based material as disclosed in WO2020127743A1 or WO2020127749A1, the content of which with regard to the components and method of preparation is herewith included by reference.

According to an embodiment, the polymeric material is a copolymer such as poly(urea-urethane), poly(ester amide)s and mixtures thereof.

According to an embodiment, the polymeric material is present in an amount less than 10%, preferably less than 8%, preferably less than 6%, preferably less than 4%, more preferably less than 3%, even more preferably less than 2% by weight based on the total weight of the microcapsule. According to a particular embodiment, the polymeric material is present in an amount between 1 and less than 10%, preferably between 1 and less than 8%, preferably between 1 and less than 6%, preferably between 1 and less than 4%, more preferably between 1 and less than 3%, even more preferably between 1 and less than 2% by weight based on the total weight of the microcapsule. Indeed, it has been underlined that even with a reduced amount of the polymeric material forming the wall, microcapsules still show good stability in consumer products.

### Plant-Based Coacervate

According to the invention, the coacervate is made of (comprises) a first and a second polyelectrolytes wherein the first polyelectrolyte comprises a plant-protein.

According to an embodiment, the plant-protein (first polyelectrolyte) is chosen in the group consisting of potato protein, chickpea protein, pea protein, faba bean protein, barley protein, oat protein, soy protein, algae protein, wheat gluten protein, lupin protein, canola protein, hemp protein, rice protein, sunflower seed protein, and mixtures thereof.

The first polyelectrolyte can comprise a plant-protein and a non-plant-protein such as fungal protein, mycoprotein, and mixtures thereof.

According to another embodiment, the first polyelectrolyte consists of a plant-protein.

According to an embodiment, the first polyelectrolyte carries a net positive charge when the pH is less than 8 while the second polyelectrolyte carries a net negative charge when the pH is greater than 2.

According to an embodiment, the solubility of the protein, preferably plant-protein, is greater than 10%. According to an embodiment, the solubility of the protein, preferably plant-protein, is greater than 20%. According to an embodiment, the solubility of the protein, preferably plant-protein, is greater than 30%. According to an embodiment, the solubility of the protein, preferably plant-protein, is greater than 40%. According to an embodiment, the solubility of the protein, preferably plant-protein, is greater than 50%. According to an embodiment, the solubility of the protein, preferably plant-protein, is greater than 60%. According to an embodiment, the solubility of the protein, preferably plant-protein, is greater than 70%. According to an embodiment, the solubility of the protein, preferably plant-protein, is greater than 80%. According to an embodiment, the solubility of the protein, preferably plant-protein, is greater than 90%. The above solubilities are given in water at room temperature (typically 20° C.) and preferably at native pH.

The protein, preferably plant-protein, used in this invention may be native, partially or completely denaturated by any suitable method. Denaturation is a process which modify the conformational structure of a protein by unfolding, i.e., it involves the disruption and possible destruction of both the secondary and tertiary structures of the protein. Indeed, denaturation implicates the breaking of many of the weak linkages, or bonds (e.g., hydrogen bonds), within a protein molecule that are responsible for the highly ordered structure of the protein in its native state. Denaturation is reversible (the proteins can regain their native state when the denaturating influence is removed) or irreversible.

Denaturation can be brought about in various ways. Proteins can be denatured by exposure to temperature, radiation or mechanical stress including shear, changes in pH (treatment with a base or an acid), treatment with oxidizing or reducing agents, inorganic salt, certain organic solvents, chaotropic agents (i.e, compounds having a positive chaotropic value—kJ Kg⁻¹ mole on the Hallsworth Scale—such as guanidine salts—e.g., guanidine carbonate, guanidine hydrochloride—, urea, calcium chloride, n-butanol, ethanol, lithium perchlorate, lithium acetate, magnesium chloride, phenol, 2-propanol, sodium dodecyl sulfate, thiourea).

The protein used in this invention can also be derivatized or modified (e.g., derivatized or chemically modified). For example, the protein can be modified by covalently attaching sugars, lipids, peptides or chemical groups such as phosphates or methyl.

According to an embodiment, the first polyelectrolyte comprises at least one polypeptide.

According to a particular embodiment, the plant-protein is potato protein.

Potato proteins are typically extracted from potato tuber (Solanum tuberosum). According to an embodiment, the potato protein is a native potato protein and preferably comprises or consisting of patatin.

A second polyelectrolyte, which is preferably selected among polysaccharides or another polymer bearing charges of opposite sign compared to the first polyelectrolyte. Generally, the second polyelectrolyte is negatively charged for pH >2.

According to an embodiment, the second polyelectrolyte is chosen in the group consisting of gum arabic, alginate salts, cellulose derivatives, guar gum, pectinate salts, pectin, carrageenan, polyacrylic and methacrylic acid, cellulose derivatives, xanthan gum, microbial exopolysaccharide, and mixtures thereof.

Among cellulose derivatives, one may cite for example carboxymethylcellulose (having preferably a molecular weight between 35 000 to 50 000 Da), hydroxypropylmethylcellulose (HPMC) or a mixture thereof.

According to a particular embodiment, the second polyelectrolyte is gum Arabic. The weight ratio between the first polyelectrolyte and the second polyelectrolyte is preferably comprised between 0.2 to 5, preferably 0.5 to 2, more preferably between 1 to 2.

According to a particular embodiment, the weight ratio between the first polyelectrolyte and the second polyelectrolyte is 1.

According to a particular embodiment, the weight ratio between the first polyelectrolyte and the second polyelectrolyte is 1.5.

According to an embodiment, the microcapsule comprises:
a core comprising a perfume oil, and
a shell comprising a polyurea and a coacervate comprising potato protein and gum arabic.
   According to an embodiment, the microcapsule comprises:
a core comprising a perfume oil, and
a shell comprising a polyurea and a coacervate comprising canola protein and gum arabic.

According to a preferred embodiment, the coacervate is hardened chemically using a suitable cross-linker such as glutaraldehyde, glyoxal, formaldehyde, polyphenol (such as tannic acid), polyanhydrides or genipin, preferably used in an amount comprised between 2% and 60%, preferably between 2% and 30% by weight based on the first polyelectrolyte.

Polyanhydride cross-linker can be poly(ethylene-maleic anhydride) or poly(methyl vinyl ether-maleic anhydride).

According to a particular embodiment, the coacervate is hardened chemically using glutaraldehyde as a cross-linker.

According to another particular embodiment, the coacervate is hardened enzymatically using an enzyme such as transglutaminase.

According to another embodiment, the coacervate is not cross-linked.

### Optional Components

According to an embodiment, the microcapsule slurry comprises auxiliary ingredients selected from the group of thickening agents/rheology modifiers, preservatives agents, antimicrobial agents, opacity-building agents, mica particles, salt, pH stabilizers/buffering ingredients, preferably in an amount comprised between 0 and 15%, more preferably between 0.1 and 10%, even more preferably between 0.05 and 5% by weight based on the total weight of the slurry.

Among the different thickening agents, one may cite for example anionic, cationic, non-ionic or zwitter-ionic copolymers, for instance, but not limited to polyacrylamide, polyacrylate, polyacryloyldimethyl taurate, polyquaternium-37, or carbomer and mixtures thereof. According to a particular embodiment, the thickening agent is xanthan gum, guar gum, diutan gum or mixtures thereof.

Among the different preservatives agents, one may cite for example sodium benzoate, benzoic acid, benzisothiazolinone, methylchloroisothiazolinone, methylisothiazolinone, chlorhexidine digluconate, sodium hydroxymethylglycinate, parabens, triclosan, phenoxyethanol, caprylhydroxamic acid, potassium sorbate, lactic acid, E-polylysine, caprylyl glycol, caprylhydroxamic acid, glycerin, glyceryl caprylate, ethylhexylglycerin and mixtures thereof.

According to another embodiment, the microcapsule slurry of the invention comprises additional free (i.e non-encapsulated) perfume, preferably in an amount comprised between 5 and 50% by weight based on the total weight of the slurry.

### Outer Coating

According to a particular embodiment of the invention, microcapsules according to the invention comprise an outer coating material selected from the group consisting of a polysaccharide, a cationic polymer, a polysuccinimide derivative (as described for instance in WO2021185724) and mixtures thereof to form an outer coating to the microcapsule.

Polysaccharide polymers are well known to a person skilled in the art. Preferred non-ionic polysaccharides are selected from the group consisting of locust bean gum, xyloglucan, guar gum, hydroxypropyl guar, hydroxypropyl cellulose and hydroxypropyl methyl cellulose, pectin and mixtures thereof.

According to a particular embodiment, the coating consists of a cationic coating.

Cationic polymers are also well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 2M Dalton.

According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride.

As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry. It is clearly understood by a person skilled in the art that only part of said added polymers will be incorporated into/deposited on the microcapsule shell.

In a particular embodiment, the shell material is a biodegradable material.

In a particular embodiment, the shell has a biodegradability of at least 60%, preferably at least 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and optionally coating may have a biodegradability of at least 40%, preferably at least 60%, preferably at least 65%, 70%, 75%, 80%, 85%, 90%, 95% or 98% within 60 days according to OECD301F.

In a particular embodiment, the oil core, preferably perfume oil, has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

### Multiple Microcapsule System

According to an embodiment, the microcapsules of the invention (first microcapsule slurry) can be used in combination with a second microcapsules slurry.
Another object of the invention is a microcapsule delivery system comprising:
the microcapsule slurry of the present invention as a first microcapsule slurry, and
a second microcapsule slurry, wherein the microcapsules contained in the first microcapsule slurry and the second microcapsule slurry differ in their hydrophobic material and/or their wall material and/or content of wall material and/or in the curing conditions to form the wall material and/or in their coating material.
   According to a particular embodiment, the microcapsule delivery system is in the form of a slurry.

The wall of the second type of microcapsules can vary. As non-limiting examples, the polymer shell of the second type of microcapsules comprises a material selected from the group consisting of polyurea, polyurethane, polyamide, polyhydroxyalkanoates, polyacrylate, polyesters, polyaminoesters, polyepoxides, polysiloxane, polycarbonate, polysulfonamide, urea formaldehyde, melamine formaldehyde resin, melamine formaldehyde resin cross-linked with polyisocyanate or aromatic polyols, melamine urea resin, melamine glyoxal resin, gelatin/gum arabic shell wall, and mixtures thereof.

The second type of microcapsule can comprise an oil-based core comprising a hydrophobic active, preferably perfume, and a composite shell comprising a first material and a second material, wherein the first material and the second material are different, the first material is a coacervate, the second material is a polymeric material. In a particular embodiment, the weight ratio between the first material and the second material is comprised between 50:50 and 99.9:0.1. In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives, guar gum, pectinate salts, carrageenan, polyacrylic and methacrylic acid or xanthan gum, or yet plant gums such as acacia gum (Gum Arabic), most preferably Gum Arabic. The coacervate first material can be hardened chemically using a suitable cross-linker such as glutaraldehyde, glyoxal, formaldehyde, tannic acid or genipin or can be hardened enzymatically using an enzyme such as transglutaminase. The second polymeric material can be selected from the group consisting of polyurea, polyurethane, polyamide, polyester, polyacrylate, polysiloxane, polycarbonate, polysulfonamide, polymers of urea and formaldehyde, melamine and formaldehyde, melamine and urea, or melamine and glyoxal and mixtures thereof, preferably polyurea and/or polyurethane. The second material is preferably present in an amount less than 3 wt. %, preferably less than 1 wt. % based on the total weight of the second type of microcapsule slurry.

As non-limiting examples, the shell of the second type of microcapsules can be aminoplast-based, polyurea-based or polyurethane-based. The shell of the second type of microcapsules can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to an aspect, the shell of the second type of microcapsules comprises an aminoplast copolymer, such as melamine-formaldehyde or urea-formaldehyde or cross-linked melamine formaldehyde or melamine glyoxal.

According to another aspect the shell of the second type of microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted. According to a particular aspect, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another aspect, the emulsifier is an anionic or amphiphilic biopolymer, which may be, in one aspect, chosen from the group consisting of gum Arabic, soy protein, gelatin, sodium caseinate and mixtures thereof.

According to another embodiment, the microcapsule wall material of the second type of microcapsules may comprise any suitable resin and especially including melamine, glyoxal, polyurea, polyurethane, polyamide, polyester, etc. Suitable resins include the reaction product of an aldehyde and an amine, suitable aldehydes include, formaldehyde and glyoxal. Suitable amines include melamine, urea, benzoguanamine, glycoluril, and mixtures thereof. Suitable melamines include, methylol melamine, methylated methylol melamine, imino melamine and mixtures thereof. Suitable ureas include, dimethylol urea, methylated dimethylol urea, urea-resorcinol, and mixtures thereof. Suitable materials for making may be obtained from one or more of the following companies Solutia Inc. (St Louis, Missouri U.S.A.), Cytec Industries (West Paterson, New Jersey U.S.A.), Sigma-Aldrich (St. Louis, Missouri U.S.A.). According to another embodiment, the second type of microcapsules is a one-shell aminoplast core-shell microcapsule obtainable by a process comprising the steps of:
1) admixing a perfume oil with at least a polyisocyanate having at least two isocyanate functional groups to form an oil phase;
2) dispersing or dissolving into water an aminoplast resin and optionally a stabilizer to form a water phase;
3) preparing an oil-in-water dispersion, wherein the mean droplet size is comprised between 1 and 100 microns, by admixing the oil phase and the water phase;
4) performing a curing step to form the wall of said microcapsule; and
5) optionally drying the final dispersion to obtain the dried core-shell microcapsule.
   According to an embodiment, the second type of microcapsules is a formaldehyde-free capsule. Atypical process for the preparation of aminoplast formaldehyde-free microcapsules slurry comprises the steps of
1) preparing an oligomeric composition comprising the reaction product of, or obtainable by reacting together:
   a. a polyamine component in the form of melamine or of a mixture of melamine and at least one C₁-C₄ compound comprising two NH2 functional groups;
   b. an aldehyde component in the form of a mixture of glyoxal, a C₄₋₆ 2,2-dialkoxy-ethanal and optionally a glyoxalate, said mixture having a molar ratio glyoxal/C₄₋₆ 2,2-dialkoxy-ethanal comprised between 1/1 and 10/1; and
   c. a protic acid catalyst;
2) preparing an oil-in-water dispersion, wherein the droplet size is comprised between 1 and 600 microns, and comprising:
   a. an oil;
   b. a water medium:
   c. at least an oligomeric composition as obtained in step 1;
   d. at least a cross-linker selected amongst:
      i. C₄-C₁₂ aromatic or aliphatic di- or tri-isocyanates and their biurets, triurets, trimmers, trimethylol propane-adduct and mixtures thereof; and/or
      ii. a di- or tri-oxiran compounds of formula:
         A-(oxiran-2-ylmethyl)ₙ
         wherein ₙ stands for 2 or 3 and 1 represents a C₂-C₆ group optionally comprising from 2 to 6 nitrogen and/or oxygen atoms;
   e. optionally a C₁-C₄ compounds comprising two NH₂ functional groups;
3) Heating the dispersion; and
4) Cooling the dispersion.

In another particular embodiment, the second type of microcapsule comprises
an oil-based core comprising a hydrophobic active, preferably perfume,
optionally an inner shell made of a polymerized polyfunctional monomer;
a biopolymer shell comprising a protein, wherein at least one protein is cross-linked.

According to a particular embodiment, the protein is chosen in the group consisting of milk proteins, caseinate salts such as sodium caseinate or calcium caseinate, casein, whey protein, hydrolyzed proteins, gelatins, gluten, pea protein, soy protein, silk protein and mixtures thereof, preferably sodium caseinate, most preferably sodium caseinate

According to a particular embodiment, the protein comprises sodium caseinate and a globular protein, preferably chosen in the group consisting of whey protein, beta-lactoglobulin, ovalbumine, bovine serum albumin, vegetable proteins, and mixtures thereof.

The protein is preferably a mixture of sodium caseinate and whey protein.

According to a particular embodiment, the biopolymer shell comprises a crosslinked protein chosen in the group consisting of sodium caseinate and/or whey protein.

According to a particular embodiment, the second type of microcapsules slurry comprises at least one microcapsule made of:
an oil-based core comprising the hydrophobic active, preferably perfume;
an inner shell made of a polymerized polyfunctional monomer; preferably a polyisocyanate having at least two isocyanate functional groups
a biopolymer shell comprising a protein, wherein at least one protein is cross-linked; wherein the protein contains preferably a mixture comprising sodium caseinate and a globular protein, preferably whey protein.
optionally at least an outer mineral layer.

According to an embodiment, sodium caseinate and/or whey protein is (are) cross-linked protein(s).

The weight ratio between sodium caseinate and whey protein is preferably comprised between 0.01 and 100, preferably between 0.1 and 10, more preferably between 0.2 and 5.

In another particular embodiment, the second type of microcapsules is a polyamide core-shell polyamide microcapsule comprising:
an oil-based core comprising comprising a hydrophobic active, preferably perfume, and
a polyamide shell comprising or being obtainable from:
   an acyl chloride,
   a first amino compound,
   a second amino compound,
   optionally, a carbohydrate

According to a particular embodiment, the second type of microcapsules comprises:
an oil-based core comprising a hydrophobic active, preferably perfume, and
a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   a stabilizer, preferably a biopolymer, preferably in an amount comprised between 0 and 90%, preferably between 0.1 and 75%, more preferably between 1 and 70%,
   optionally, a carbohydrate.

According to a particular embodiment, the second type of microcapsules comprises:
an oil-based core comprising a hydrophobic active, preferably perfume, and
a polyamide shell comprising or being obtainable from:
an acyl chloride,
a first amino-compound being an amino-acid, preferably chosen in the group consisting of L-Lysine, L-Arginine, L-Histidine, L-Tryptophane and/or mixtures thereof.
a second amino-compound, preferably chosen in the group consisting of ethylene diamine, diethylene triamine, cystamine and/or mixtures thereof, and
a biopolymer, preferably chosen in the group consisting of potato protein, chickpea protein, pea protein, algae protein, faba bean protein, barley protein, oat protein, wheat gluten protein, lupin protein, soy protein, rice protein, whey protein, white egg albumin, casein, sodium caseinate, gelatin (preferably fish gelatin), bovine serum albumin, hydrolyzed soy protein, hydrolyzed sericin, pseudo collagen, silk protein, sericin powder, gelatin and mixtures thereof,
optionally a carbohydrate, preferably selected from the group consisting of anionic salt of alginic acid, preferably alginic acid sodium salt, pectin, lignin, anionic modified starch, carboxymethylcellulose, carrageenan and mixtures thereof.

According to another aspect, the shell of the second type of microcapsules is polyurea- or polyurethane-based. Examples of processes for the preparation of polyurea and polyurethane-based microcapsule slurry are for instance described in International Patent Application Publication No. WO2007/004166, European Patent Application Publication No. EP 2300146, and European Patent Application Publication No. EP25799. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a water phase;
c) Adding the oil phase to the water phase to form an oil-in-water dispersion, wherein the mean droplet size is comprised between 1 and 500 μm, preferably between 5 and 50 μm; and
d) Applying conditions sufficient to induce interfacial polymerisation and form microcapsules in form of a slurry.

### Microcapsule Powder

Another object of the invention is a microcapsule powder obtained by submitting the microcapsule slurry to a drying, like spray-drying, to provide the microcapsules as such, i.e. in a powdery form. It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, gum Arabic, vegetable gums, pectins, xanthans, alginates, carrageenans or cellulose derivatives to provide microcapsules in a powder form.

However, one may cite also other drying method such as the extrusion, plating, spray granulation, the fluidized bed, or even a drying at room temperature using materials (carrier, desiccant) that meet specific criteria as disclosed in WO2017/134179.

According to a particular embodiment, the carrier material contains free perfume oil which can be the same or different from the perfume from the core of the microcapsules.

Another object of the invention is a solid particle comprising:
a carrier material,
microcapsules as defined above entrapped in said carrier material, and
optionally free perfume entrapped in said carrier material.

In a particular embodiment, the carrier material comprises a monomeric, oligomeric or polymeric carrier material, or mixtures of two or more of these.

An oligomeric carrier is a carrier wherein 2-10 monomeric units are linked by covalent bonds. For example, if the oligomeric carrier is a carbohydrate, the oligomeric carrier may be sucrose, lactose, raffinose, maltose, trehalose, fructo-oligosaccharides.

Examples of a monomeric carrier materials are glucose, fructose, mannose, galactose, arabinose, fucose, sorbitol, mannitol, for example.

Polymeric carriers have more than 10 monomeric units that are linked by covalent bonds.

In a particular embodiment, the carrier may be a polymeric carrier material. Non-limiting examples of polymeric carrier material includes polyaspartate, modified polysuccinimides, lignin and its derivatives, polyoxazoline, polyhydroxyalcanoates, polyphenols, natural and synthetic clays, polyvinyl acetates, polyvinyl alcohol, dextrines, maltodextrines, glucose syrups, natural or modified starch, polysaccharides, carbohydrates, chitosan, gum Arabic, polyethylene glycol, polyvinyl pyrrolidone, polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carrageenans or cellulose derivatives, such as carboxymethyl methylcellulose, methylcellulose or hydroxyethyl cellulose; chitin, proteins (animal and vegetal), polyaspartate, polysuccinimide and its derivatives, polyesters, polyaminoesters, polyhydroxyalkanoates, polycarbonates and mixtures thereof. Preferably the polymeric carrier material comprises natural or modified starch, maltodextrins, carbohydrates, chitin, proteins (animal and vegetal), polyaspartate, polysuccinimides and its derivatives, polyesters, polyaminoesters, polyhydroxyalkanoates, polycarbonates and mixtures thereof.

According to an embodiment, the carrier material is chosen in the group consisting of polyvinyl acetate, polyvinyl alcohol, dextrins, natural or modified starch, vegetable gums, pectins, xanthans, alginates, carrageenans, cellulose derivatives and mixtures thereof.

Solid particle as defined above and microcapsule powder can be used indifferently in the present invention.

### Process for Preparing Microcapsules

Microcapsules of the present invention can be prepared by different processes.

### Embodiment 1

The present invention also relates to a process for preparing core-shell microcapsule comprising the steps of:
(i) providing a hydrophobic phase comprising a hydrophobic material and at least a polyfunctional monomer;
(ii) mixing a first polyelectrolyte and a second polyelectrolyte in a dispersing phase under conditions sufficient to not form a suspension of complex coacervate; wherein the first polyelectrolyte comprises a plant-protein,
(iii) adding the hydrophobic phase into the dispersing phase to form a two phases dispersion and applying sufficient condition to form a coacervate,
(iv) providing conditions sufficient to induce interfacial polymerization to form a core-shell microcapsule slurry.

### Embodiment 2

The present invention also relates to a process for preparing core-shell microcapsule comprising the steps of:
(i) mixing a first polyelectrolyte and second polyelectrolyte in a dispersing phase under conditions sufficient to not form a suspension of complex coacervate; wherein the first polyelectrolyte comprises a plant-protein,
(ii) providing a hydrophobic phase comprising a hydrophobic material and at least a polyfunctional monomer;
(iii) adding the hydrophobic phase into the dispersing phase to form a two phases dispersion and applying sufficient condition to form a coacervate,
(iv) providing conditions sufficient to induce interfacial polymerization to form a core-shell microcapsule slurry.

### Embodiment 3

The present invention also relates to a process for preparing core-shell microcapsule comprising the steps of:
(i) Adding a hydrophobic phase comprising a hydrophobic material and at least a polyfunctional monomer into a dispersing phase comprising a first polyelectrolyte; wherein the first polyelectrolyte comprises a plant-protein to form a two-phases dispersion;
(ii) adding a second polyelectrolyte in the two-phases dispersion
(iii) applying sufficient condition to form a coacervate,
(iv) providing conditions sufficient to induce interfacial polymerization to form a core-shell microcapsule slurry.

According to an embodiment, the pH of the solution containing both polyelectrolytes (step ii) for embodiment 1; step i) for embodiment 2); step ii) in embodiment 3) is comprised between 1 and less than 2.5, preferably between 1.5 and 2.2, so the polyelectrolytes cannot interact to form a coacervate.

According to an embodiment, the pH of the solution containing both polyelectrolytes (step ii) for embodiment 1; step i) for embodiment 2); step ii) in embodiment 3) is comprised between 5 and 8, preferably between 5.5 and 7, so the polyelectrolytes cannot interact to form a coacervate.

The coacervation takes place in step iii), where the pH of the solution is typically comprised between 2.5 and 4.2, preferably between 2.8 and 4.

The interfacial polymerization can be carried out typically at a temperature between 50° C. and 80° C. under stirring for 2 to 40 hours to complete the reaction and form microcapsules in the form of a slurry. However, the interfacial polymerization can be carried out at room temperature, typically between 2° and 30° C.

According to an embodiment, the dispersing phase comprises, preferably consists of water.

According to an embodiment, the dispersing phase is a water phase.

According to an embodiment, the two-phases dispersion is an oil-in-water emulsion.

According to an embodiment, the dispersing phase comprises water and an alcohol such as glycerol, 1,4-butanediol, ethylene glycol and mixtures thereof.

According to an embodiment, the hydrophobic phase is an oil-phase.

According to an embodiment, the hydrophobic phase and/or the dispersing phase comprises an emulsifier, a surfactant or mixtures thereof. The surfactant may be non-ionic, anionic, cationic or zwitterionic.

Emulsifiers and/or surfactants can be chosen in the list consisting of polyvinylalcohol, sodium stearoyl lactylate, esters of mono- and diglycerides, lecithins, saponins, ascorbyl palmitate, sugar esters, sucrose esters, sucroglycerides, polyglycerin polyricinoleate, propylene glycol esters of fatty acids, sorbitan esters such as sorbitan tristearate, sorbitan monooleate, sorbitan monolaurate, sorbitan monostearate, sorbitan sesquioleate, sorbitan trioleate, sorbitan monopalmitate, polysorbates such as polyoxyethylene (20) sorbitan monolaurate (polysorbate 20), polysorbate 40, polysorbate 60, polysorbate 80, Brij 35 (polyoxyethylene lauryl ether) or Brij 93 (Polyethylene glycol oleyl ether), sodium dodecyl sulfate, taurides, sulfoacetates, alkyl ether sulfates, fatty alcohol sulfates, secondary alkyl sulfonates, alkylbenzol sulfonates, alkylcarboxylates and mixtures thereof.

Emulsifiers and/or surfactants are typically used in an amount comprised between 0.001 and 5% based on the hydrophobic phase and/or the dispersing phase.

According to an embodiment the process steps are performed such that foam formation is suppressed or reduced. Such an embodiment comprises for example the use of appropriate mixing devices and vessels, adapting the stirring speed and vessel geometry to minimize gas bubble formation, filling and mixing steps, or the use of appropriate dosing devices or dosing methods, including membranes or microcapillary devices. Suppression or reduction of foam formation may also be achieved by addition of antifoaming (de-foaming) agents, which may be introduced in any of the process steps.

Non limiting examples of antifoaming agents include mineral oil, oil, aliphatic ester, alcohol, amide, phosphate, metallic soap, organosilicon and mixtures thereof.

It should be understood that the antifoaming agent is not part of the shell.

When used, the antifoaming agent is preferably used in an amount comprised between 0.05 and 1% by weight based on dispersing phase.

According to a particular embodiment, no amine or polyamine susceptible to polymerize with the polyfunctional monomer is added at any stage of the process.

The dispersion of the oil phase into the water phase can be carried out by different well-known techniques. As non-limiting methods, one may cite a static mixer (SMX, Sulzer, Switzerland), vibrating mixers, stirred tank reactor, Ultrasonic homogenizer, membrane emulsification device (Micropure, UK), high pressure homogenizer (APV, SPX Flow, USA), or a microfluidic emulsification device (Micronit, Netherland).

According to an embodiment, the polyfunctional monomer is chosen in the group consisting of at least one isocyanate, anhydride or maleic anhydride, acyl chloride, epoxide, (meth) acrylate monomers, alkoxysilane, and mixtures thereof.

According to a particular embodiment, the monomer added in step i) or ii) is at least one polyisocyanate having at least two isocyanate functional groups.

Suitable polyisocyanates used according to the invention include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the polyfunctional monomer is an acyl chloride.

According to a particular embodiment, the acyl chloride has the following formula (I)

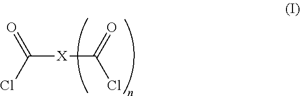

wherein n is an integer varying between 1 and 8, preferably between 1 and 6, more preferably between 1 and 4, and
wherein X is an (n+1)-valent C₂ to C₄₅ hydrocarbon group optionally comprising at least one group selected from (i) to (xi), particularly from (i) to (vi)

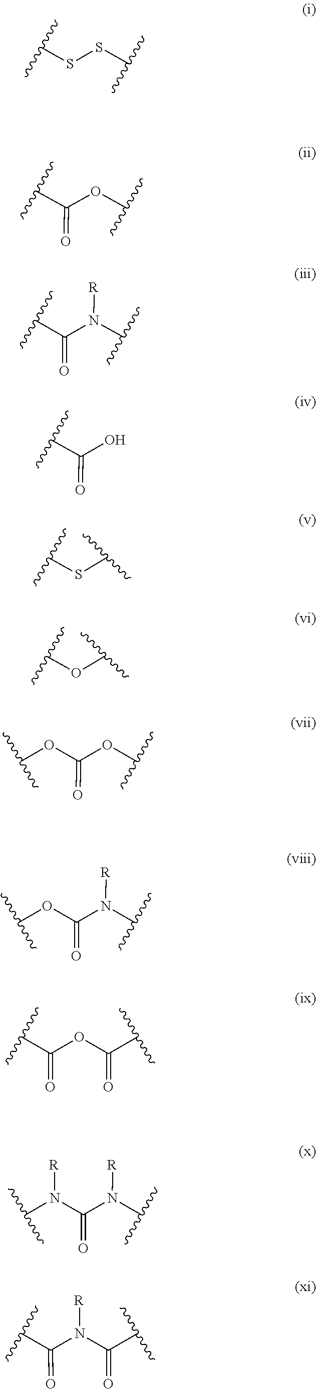

wherein R is a hydrogen atom or an alkyl group such as a methyl or an ethyl group, preferably a hydrogen atom.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynil group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises heteroatoms to form ether, thioether, amine, nitrile or carboxylic acid groups and derivatives (including for example esters, acids, amide). These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain or ring.

According to a particular embodiment, the acyl chloride is chosen from the group consisting of benzene-1,3,5-tricarbonyl trichloride (trimesoyl trichloride), benzene-1,2,4-tricarbonyl trichloride, benzene-1,2,4,5-tetracarbonyl tetrachloride, cyclohexane-1,3,5-tricarbonyl trichloride, isophthalyol dichloride, diglycolyl dichloride, terephthaloyl chloride, fumaryl dichloride, adipoyl chloride, succinic dichloride, propane-1,2,3-tricarbonyl trichloride, cyclohexane-1,2,4,5-tetracarbonyl tetrachloride, 2,2′-disulfanediyldisuccinyl dichloride, 2-(2-chloro-2-oxo-ethyl)sulfanylbutanedioyl dichloride, (4-chloro-4-oxobutanoyl)-L-glutamoyl dichloride, (S)-4-((1,5-dichloro-1,5-dioxopentan-2-yl)amino)-4-oxobutanoic acid, 2,2-bis[(4-chloro-4-oxo-butanoyl)oxymethyl]butyl 4-chloro-4-oxo-butanoate, [2-[2,2-bis[(4-chloro-4-oxo-butanoyl)oxymethyl]butoxymethyl]-2-[(4-chloro-4-oxo-butanoyl)oxymethyl]butyl]4-chloro-4-oxo-butanoate, 2,2-bis[(2-chlorocarbonylbenzoyl)oxymethyl]butyl 2-chlorocarbonyl-benzoate, [2-[2,2-bis[(2-chlorocarbonylbenzoyl)oxymethyl]butoxymethyl]-2-[(2-chlorocarbonylbenzoyl)oxymethyl]butyl]2-chlorocarbonylbenzoate, 4-(2,4,5-trichlorocarbonylbenzoyl)oxybutyl 2,4,5-trichlorocarbonyl-benzoate, propane-1,2,3-triyl tris(4-chloro-4-oxobutanoate), propane-1,2-diyl bis(4-chloro-4-oxobutanoate) and mixtures thereof.

According to an embodiment, the polyfunctional monomer used in the process of the invention is present in amounts representing from 0.1 and 15%, preferably from 0.5 and 3% by weight based on the total amount of the oil phase.

According to anyone of the preceding embodiments, in addition to the polyfunctional monomer present in the oil phase, the aqueous phase may comprise an amino resin.

Another object of the present invention is a core-shell microcapsule slurry obtainable by the processes disclosed above.

### Perfuming Composition and Consumer Products

The microcapsules of the invention can be used in combination with active ingredients.

An object of the invention is therefore a composition comprising:
(i) microcapsules or microcapsule slurry as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.
   The capsules of the invention show a good performance in terms of stability in challenging medium.

Another object of the present invention is a perfuming composition comprising:
(i) microcapsules or microcapsule slurry as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.

As liquid perfumery carriers one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds. Co-ingredients may be chosen in the group consisting of 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof or a mixture thereof.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules or microcapsule slurry as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) a microcapsule slurry or microcapsules as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) a microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care, surface care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsules than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc.). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a perfume, such as a fine perfume, a cologne, an after-shave lotion, a body-splash; a fabric care product, such as a liquid or solid detergent, tablets and unit dose (single or multi-chambers), a fabric softener, a dryer sheet, a fabric refresher, an ironing water, or a bleach; a personal-care product, such as a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules or microcapsule slurry as defined above or the perfuming composition as defined above,
   wherein the consumer product is in the form of a personal care composition.

Personal care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product);

Another object of the invention is a consumer product comprising:
a home care or a fabric care active base, and
microcapsules or microcapsule slurry as defined above or the perfuming composition as defined above,
   wherein the consumer product is in the form of a home care or a fabric care composition.

Home care or fabric care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules or microcapsule slurry of the present invention, these percentages being defined by weight relative to the total weight of the consumer product. Of course, the above concentrations may be adapted according to the benefit effect desired in each product.

An object of the invention is a consumer product, preferably a home care or a fabric care consumer product comprising the microcapsules or the microcapsule slurry as defined above, wherein the consumer product has a pH less than 7.

An object of the invention is a consumer product, preferably a home care or a fabric care consumer product comprising the microcapsules or the microcapsule slurry as defined above, wherein the consumer product has a pH equals or greater than 7.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ) (1,2-stearoyl-3-trimethylammonium-propane (chloride salt)), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry or microcapsules as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyl trimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry or microcapsules as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsule slurry or microcapsules as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of or microcapsule slurry or microcapsules as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Example 1

### Potato Protein-Based Composite Microcapsules

### Protocol for Preparing Microcapsules A

Potato protein solution (Solanic® 200, Origin: Avebe) was prepared in DI water (native pH 6.8).
Gum Arabic solution (Superstab™, origin: Nexira) was prepared in DI water.
The potato protein and gum Arabic solutions were adjusted at a pH of 2 using concentrate HCl (37%) before mixing them to obtain an aqueous phase.
An oil phase comprising polyisocyanate (Takenate®—Trimethylol propane-adduct of xylylene diisocyanate, origin: Mitsui Chemicals, Inc., Japan, 75% solution of polyisocyanate in ethyl acetate) and a perfume oil (see Table 1) was prepared and then added to the aqueous phase (potato protein/Gum Arabic solution (pH˜2)) under Ultra-Turrax/13500 rpm−1 minute to obtain an oil-in-water emulsion.
The pH was then increased to pH 3 by adding NaOH 1M (0.73 g) under stirring.
After checking by optical microscopy the coacervation took place, glutaraldehyde was added and the suspension was stirred for 4 hours at RT.

TABLE 1
Perfume oil A composition

| Ingredients | % in oil |
|---|---|
| Ethyl 2-methyl-pentanoate | 3.2% |
| Eucalyptol | 7.8% |
| 2,4-Dimethyl-3-cyclohexene-1-carbaldehyde | 0.75% |
| Aldehyde C10 | 0.75% |
| Citronellyl Nitrile | 4.3% |
| Isobornyl acetate | 3% |
| 2-tert-butyl-1-cyclohexyl acetate | 9.8% |
| Citronellyl Acetate | 1.3% |
| 2-Methylundecanal | 3% |
| Diphenyloxide | 0.8% |
| Aldehyde C12 | 1.3% |
| Dicyclopentadiene acetate | 9.85% |
| Ionone beta | 3.3% |
| Undecalactone gamma | 18.75% |
| Hexyl Salicylate | 15.9% |
| Benzyl Salicylate | 16.2% |

TABLE 2
Microcapsule composition

| | m, g | active, g |
|---|---|---|
| Potato protein ¹⁾ | 30.7 | 0.60 |
| Gum Arabic 2% ²⁾ | 30.0 | 0.60 |
| Perfume | 8.0 | 8.0 |
| Polyisocyanate ³⁾ | 0.24 | 0.18 |
| glutaraldehyde 50%⁴⁾ | 0.63 | 0.3 |
| Total | | |

¹⁾ Solanic 200 2% solution, origin: Avebe
²⁾ Superstab ™, origin: Nexira
³⁾ Takenate ® D110N, origin: Mitsui Chemicals, Inc
⁴⁾Origin: Merck-Sigma Aldrich

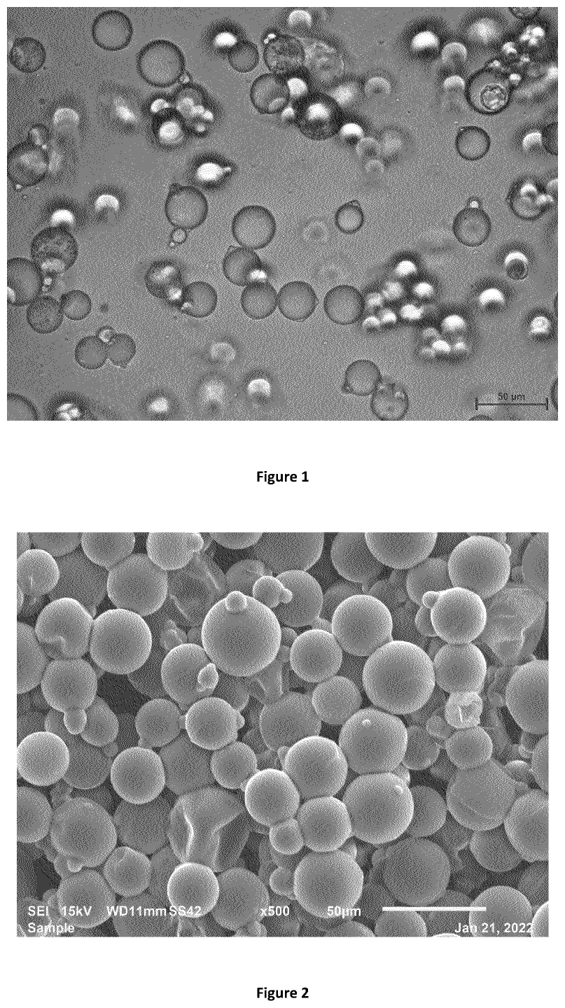

FIG. 1 and FIG. 2 represent respectively the optical microscopy and SEM microscopy of microcapsules.

### Example 2

### Potato Protein-Based Composite Microcapsules

Microcapsules B were prepared according to protocol of example 1, except that at the end of the process, the suspension was heated for 30 minutes at 85° C.

### Example 3

### Canola Protein-Based Composite Microcapsules

### Protocol for Preparing Microcapsules C

50 g of Canola protein (CP) (CanolaPro®, origin: DSM) 4% solution was prepared in deionized water (native pH=6.5).
50 g of Gum arabic (GA) (Superstab™, origin: Nexira) 4% solution was prepared in deionized water (pH=6)
Both solutions were mixed together and concentrate HCl (37%) (0.36 g) was added to lower the pH to 2.4.
An oil solution made of 30 g perfume and 0.75 g Takenate® D110N was added to the biopolymer solution and sheared using an ultra turrax 2 minutes at 9500 rpm to form an oil-in-water emulsion.
The pH of the emulsion was then adjusted to 4.09 by adding dropwise NaOH 1M solution (2.3 g) under mechanical stirring.
Coacervation of the CP/GA complex around the oil droplets was checked by optical microscopy.
Then, 0.22 g glutaraldehyde 50% solution was added to the thus-obtained suspension and the slurry was stirred at 800 rpm for 3.5 hours at room temperature, followed by 1 hour heating at 75° C., in the same stirring conditions.

TABLE 3
Microcapsule composition

| | real, g | active, g |
|---|---|---|
| Canola Protein 4% ¹⁾ | 50 | 2 |
| Gum Arabic 4% ²⁾ | 50 | 2 |
| Perfume | 30.0 | 30.0 |
| Polyisocyanate ³⁾ | 0.76 | 0.57 |
| Glutarladehyde 50% ⁴⁾ | 0.22 | 0.11 |

¹⁾ Origin: DSM
²⁾ Superstab ™, origin: Nexira
³⁾ Takenate ® D110N, origin: Mitsui Chemicals, Inc
⁴⁾ Origin: Merck-Sigma Aldrich

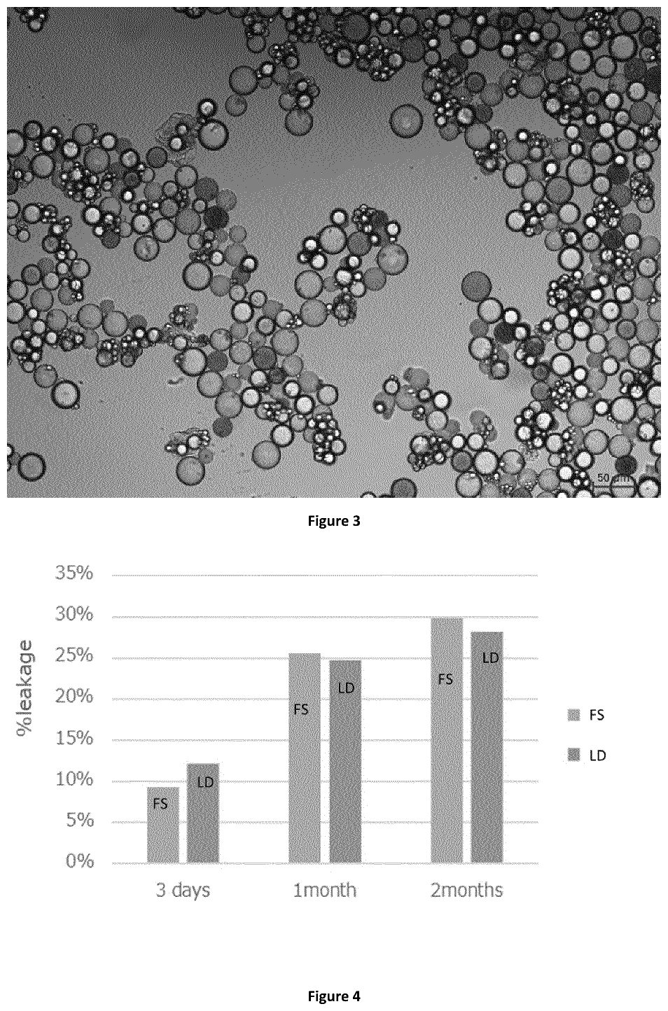

FIG. 3 represents respectively the optical microscopy and SEM microscopy of microcapsules.

### Example 4

### Stability of Invention's Microcapsules

Microcapsules of the present invention are dispersed in a fabric softener (FS) composition described in Table 4 or in a liquid detergent (LD) composition described in Table 5 to obtain a concentration of encapsulated perfume oil at 0.116%.

TABLE 4
Fabric Conditioner composition

| Product | Wt % |
|---|---|
| Stepantex VL 90A | 8.88 |
| Calcium Chloride Sol. 10% | 0.36 |
| Proxel GXL | 0.04 |
| Perfume | 1.00 |
| Water | 89.72 |
| TOTAL | 100 |

TABLE 5
Liquid detergent composition

| Ingredients | Concentration [wt %] |
|---|---|
| Sodium C₁₄₋₁₇ Alkyl Sec Sulfonate¹⁾ | 7 |
| Fatty acids, C₁₂₋₁₈ and C₁₈-unsaturated²⁾ | 7.5 |
| C_{12/14} fatty alcohol polyglycol | 17 |
| ether with 7 mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 Methacrylate | 6 |
| structuring Crosspolymer⁵⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Origin: Genencor International
⁵⁾Aculyn 88; Origin: Dow Chemical

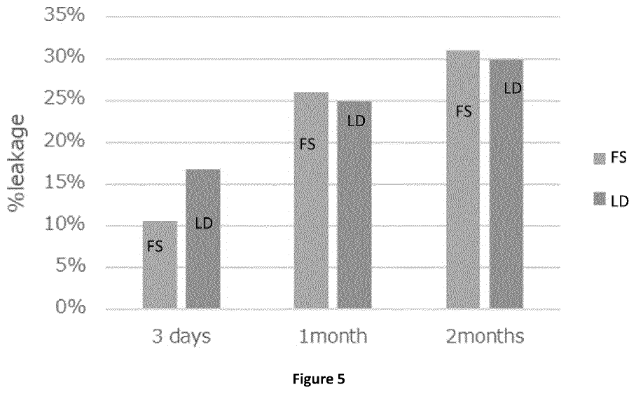

2 g of sample (base with capsules) is weighed in a 20 mL vial. 10 mL of the extraction solvent isooctane containing the internal standard 1,4-dibromobenzene at a precisely known concentration around 90 ng/uL is added to the vial. Then, it is shaked for 45 min at 40 RPM to extract the free perfume. The solvent phase is then removed.
To measure the leakage in the base the Agilent GCFID7890A is used, the injector is set at 250° C., helium is used as the carrier gas at a flow rate of 1 mL/min, the oven temperature is programmed from 120° C., held 5 minutes, increased to 170° C. at 10° C./min, increased to 220° C. at 25° C./min and then increased to 260 at 25° C./min. To finish a post run is applied at 260° C. to finish the measure.
Calibration solutions are prepared at 100, 300 and 600 ng/uL of fragrance oil in the isooctane. It is important that the fragrance oil used to prepare the calibration curve comes from the same batch used to produce the microcapsules.
Stability of microcapsules A and B are shown respectively in FIGS. 4 and 5.
It can be concluded that the microcapsules of the present invention show good stability in a fabric softener and in a liquid detergent.

### Example 5

### Olfactive Performance of Invention's Microcapsules

### Protocol

The perfume oil dosage in the fabric softener was 0.1%.
The perfume oil dosage in the liquid detergent was 0.2%.

### Washing Protocol:

Program: 40° C.—short spinning—900 rpm
Towels: 36
Unperfumed detergent: 55 g
Softener: 23 g

### Storage Protocol

Dry: Towels left to dry on a drying rack for 1 day

### Evaluation:

One set of towels per panelist: the panelists were asked to smell the dry towels and evaluate the perfume intensity on a scale between 0 (no odour) and 10 (very strong odour). They were then asked to rub the towels three times back and forth and evaluate the perfume intensity after rubbing on the same scale.

TABLE 6
Olfactive performance results

| | Intensity before | Intensity after |
|---|---|---|
| | rubbing (A) | rubbing (B) |
| Fabric conditioner Microcapsules A | 2.36 | 5.1 |
| Liquid detergent Microcapsules A | 4.2 | 6.1 |

A noticeable perfume boost after rubbing was perceived in both fabric conditioner and liquid detergent applications.

### Example 6

### Microcapsule Biodegradability

Shell extraction (following method disclosed in Gasparini and all in Molecules 2020, 25,718) The microcapsule slurry was lyophilized. The recovered solid was grinded using a crusher IKA tube-mill control for 30 sec. The resulting paste (fragrance oil+polymeric shells) was suspended in 300 mL of Ethyl acetate and the mixture was stirred for 1 h at room temperature. The solid was collected by filtration under vacuum over a gooch filter crucible (porosity 4). This extraction step was repeated 5 times to remove the maximum of fragrance oil from the shells. The powder was dried under vacuum (10 mBar) at 50° C. until the weight of the polymer, monitored by gravimetry, was constant. The resulting powder was grinded using a crusher IKA tube-mill control for 1 min 30 sec, suspended in Di water (0.5% w/w) and stirred at 300 RPM for 24 H at RT. The water was removed by filtration under vacuum over a gooch filter crucible (porosity 4) and the powder was dried at RT for 2.5 days and then under vacuum (10 mBar) at 50° C. overnight. Finally, the obtained powder was grinded using a crusher IKA tube-mill control for 1 min and 30 seconds, and extracted an additional five times with ethyl acetate as described before. The final powder was dried under vacuum (10 mBar) at 50° C. overnight. To ensure that the totality of the perfume was removed, the sample was analyzed by GC-pyrolysis and send to biodegradation measurement following the OECD301F method.
The biodegradability of the shell for microcapsules A was greater than 50% after 60 days of test.

### Example 7

### Shell Composition of the Capsules of the Present Invention Determined by Solid State NMR

NMR enabled to determine the following shell composition of microcapsules A, based on carbon signals from the individual components:
52% potato protein
28% Gum Arabic
14% polyisocyanate
6% residual (glutaraldehyde)
   Furthermore, it has been shown with the measurement of the relaxation time that all nuclei statistically have the same environment underlying the homogeneity of the shell.

| Ingredients | Emulsion A | Emulsion B | Emulsion C | Emulsion D | Emulsion E |
|---|---|---|---|---|---|
| Modified starch ¹⁾ | 2.6% | 2.6% | 2.6% | 12.5% | 2% |
| Maltodextrin ²⁾ | 26.8% | 22.8% | 19.3% | 0% | 19.1% |
| Maltose ³⁾ | 0% | 0% | 0% | 7.9% | 0% |
| Citric Acid | 0% | 0% | 0% | 1% | 0% |
| Tripotassium Citrate | 0% | 0% | 0% | 1.9% | 0% |
| Exemplified Microcapsules | 12.0% | 24% | 37.0% | 8.9% | 56.2% |
| Silica ⁴⁾ | 1.1% | 1.1% | 1.1% | 0% | 0% |
| Free Perfume B ⁵⁾ | 0% | 0% | 0% | 11% | 0% |
| Water | 57.6% | 49.6% | 40.1% | 56.9% | 22.7% |
| | Granule A | Granule B | Granule C | Granule D | Granule E |
| Modified starch ¹⁾ | 7.5% | 7.4% | 7.2% | 31.6% | 4.9% |
| Maltodextrin ²⁾ | 77.4% | 65.5% | 53.8% | 0% | 44.7% |
| Maltose | 0% | 0% | 0% | 20.9% | |
| Citric Acid | 0% | 0% | 0% | 2.6% | 0% |
| Tripotassium citrate | 0% | 0% | 0% | 4.9% | 0% |
| Encapsulated perfume B | 0% | 0% | 0% | 28.1% | 0% |
| Microcapsules I-L | 12.% | 24.1% | 36.1% | 9.8% | 48.4% |
| Silica | 3.0 | 3.0% | 2.9% | 2.0% | 2% |
| Fragrance loading in | 10.1% | 20.1% | 30% | 35.8% | 40.2% |
| powder after spray-drying | | | | | |

### Example 8

### Spray-Dried Microcapsules Preparation

Emulsions A-E having the following ingredients are prepared.

TABLE 7
Composition of Emulsions A-E and composition of granulated powder A-E after spray-drying

| Ingredients | Emulsion A | Emulsion B | Emulsion C | Emulsion D | Emulsion E |
|---|---|---|---|---|---|
| Modified starch ¹⁾ | 2.6% | 2.6% | 2.6% | 12.5% | 2% |
| Maltodextrin ²⁾ | 26.8% | 22.8% | 19.3% | 0% | 19.1% |
| Maltose ³⁾ | 0% | 0% | 0% | 7.9% | 0% |
| Citric Acid | 0% | 0% | 0% | 1% | 0% |
| Tripotassium Citrate | 0% | 0% | 0% | 1.9% | 0% |
| Exemplified Microcapsules | 12.0% | 24% | 37.0% | 8.9% | 56.2% |
| Silica ⁴⁾ | 1.1% | 1.1% | 1.1% | 0% | 0% |
| Free Perfume B ⁵⁾ | 0% | 0% | 0% | 11% | 0% |
| Water | 57.6% | 49.6% | 40.1% | 56.9% | 22.7% |
| | Granule A | Granule B | Granule C | Granule D | Granule E |
| Modified starch ¹⁾ | 7.5% | 7.4% | 7.2% | 31.6% | 4.9% |
| Maltodextrin ²⁾ | 77.4% | 65.5% | 53.8% | 0% | 44.7% |
| Maltose | 0% | 0% | 0% | 20.9% | |
| Citric Acid | 0% | 0% | 0% | 2.6% | 0% |
| Tripotassium citrate | 0% | 0% | 0% | 4.9% | 0% |
| Encapsulated perfume B | 0% | 0% | 0% | 28.1% | 0% |
| Microcapsules I-L | 12.% | 24.1% | 36.1% | 9.8% | 48.4% |
| Silica | 3.0 | 3.0% | 2.9% | 2.0% | 2% |
| Fragrance loading in | 10.1% | 20.1% | 30% | 35.8% | 40.2% |
| powder after spray-drying | | | | | |

1) Capsul™, Ingredion
2) Maltodextrin 10DE origin: Roquette
3) Maltose, Lehmann & Voss
4) Silica, Evonik
5) see table 8

TABLE 8
Composition of Perfume B

| Component | % |
|---|---|
| ACÉTATE DE 4-(1,1-DIMÉTHYLÉTHYL)-1- | 14.50 |
| CYCLOHEXYLE ¹⁾ | |
| LINALOL BJ | 10.50 |
| LILIAL ®²⁾ | 10.00 |
| ISO E SUPER ³⁾ | 10.00 |
| CITRONELLYL NITRILE | 9.00 |
| DIPHENYLOXYDE | 6.50 |
| ISOBORNYL ACETATE | 6.00 |
| BETA IONONE | 6.00 |
| TRICYCLO[5.2.1.0~2,6~]DEC-3-EN-8-YL | 5.50 |
| ACETATE (A) + TRICYCLO[5.2.1.0~2,6~]DEC- | |
| 4-EN-8-YL ACETATE (B) ⁴⁾ | |
| ETHER MT | 4.00 |
| HEDIONE ® ⁵⁾ | 4.00 |
| GERANIOL 60 | 3.00 |
| CITRAL | 2.50 |
| ALDEHYDE C 10 | 2.50 |
| ALLYL HEPTANOATE | 2.50 |
| ETHYL METHYL-2-BUTYRATE | 1.50 |
| GERANYL ACETATE | 1.00 |
| 2,4-DIMETHYL-3-CYCLOHEXENE-1-CARBALDEHYDE ⁶⁾ | 1.00 |

¹⁾ Firmenich SA, Switzerland
²⁾3-(4-tert-butylphenyl)-2-methylpropanal, Givaudan SA, Vernier, Switzerland
³⁾ 1-(octahydro-2,3,8,8-tetramethyl-2-naphtalenyl)-1-ethanone, International Flavors & Fragrances, USA
⁴⁾ Firmenich SA, Switzerland
⁵⁾ Methyl dihydrojasmonate, Firmenich SA, Switzerland
⁶⁾ Firmenich SA, Switzerland

1) Firmenich SA, Switzerland
2) 3-(4-tert-butylphenyl)-2-methylpropanal, Givaudan SA, Vernier, Switzerland
3) 1-(octahydro-2,3,8,8-tetramethyl-2-naphtalenyl)-1-ethanone, International Flavors Fragrances, USA
4) Firmenich SA, Switzerland
5) Methyl dihydrojasmonate, Firmenich SA, Switzerland
6) Firmenich SA, Switzerland
   Components for the polymeric matrix (Maltodextrin and Capsul™, or Capsul™, citric acid and tripotassium citrate) are added in water at 45-50° C. until complete dissolution.
   For emulsion D, free perfume C is added to the aqueous phase.
   Microcapsules slurry is added to the obtained mixture. Then, the resulting mixture is then mixed gently at 25° C. (room temperature).
   Granulated powder A-E are prepared by spray-drying Emulsion A-E using a Sodeva Spray Dryer (Origin France), with an air inlet temperature set to 215° C. and a throughput set to 500 ml per hour. The air outlet temperature is of 105° C. The emulsion before atomization is at ambient temperature.

### Example 9

### Liquid Scent Booster Composition

A sufficient amount of exemplified microcapsules is weighed and mixed in a liquid scent booster to add the equivalent of 0.2% perfume.

TABLE 9
Liquid scent booster composition

| | Amount (% wt) | | | | | |
|---|---|---|---|---|---|---|
| Ingredients | 1 | 2 | 3 | 4 | 5 | 6 |
| Water | 71.20% | 89.5% | 78.8% | 79.4% | 70% | 70% |
| Propylene | 20.30% | — | — | — | 20% | 20% |
| glycol | | | | | | |
| Polyethylene | 4.00% | 6% | | | | |
| glycol ethers of | | | | | | |
| decyl alcohol¹⁾ | | | | | | |
| Polyethylene | | | | | 4.00% | 4.00% |
| glycol ether of | | | | | | |
| Lauryl | | | | | | |
| Alcohol²⁾ | | | | | | |
| alkyl | | | 8.30% | 7.7% | | |
| polyglucoside | | | | | | |
| C8-C10³⁾ | | | | | | |
| Deceth-3¹⁾ | 1.50% | | | | | |
| Lauryl lactate | | | | 1% | | |
| Lauric acid | | 1.5% | 1.60% | | | |
| Glyceryl | | | | | 3.00% | 3.00% |
| Caprylate | | | | | | |
| Fragrance | 3.00% | 3.0% | 3.00% | 3.00% | 3.00% | 0% |

¹⁾Deceth-8; trademark and origin: KLK Oleo
²⁾Laureth-9
³⁾Plantacare 2000UP; trademark and origin: BASF

Different ringing gel compositions are prepared (compositions 1-6) according to the following protocol.

In a first step, the aqueous phase (water), the solvent (propylene glycol) if present and surfactants are mixed together at room temperature under agitation with magnetic stirrer at 300 rpm for 5 min.

In a second step, the linker is dissolved in the hydrophobic active ingredient (fragrance) at room temperature under agitation with magnetic stirrer at 300 rpm. The resulting mixture is mixed for 5 min.

Then, the aqueous phase and the oil phase are mixed together at room temperature for 5 min leading to the formation of a transparent or opalescent ringing gel.

### Example 10

### Liquid Detergent Composition

A sufficient amount of exemplified microcapsules is weighed and mixed in a liquid detergent to add the equivalent of 0.2% perfume.

TABLE 10
Liquid detergent composition

| | Concentration |
|---|---|
| Ingredients | [wt %] |
| Sodium C₁₄₋₁₇ Alkyl Sec Sulfonate¹⁾ | 7 |
| Fatty acids, C₁₂₋₁₈ and C₁₈-unsaturated²⁾ | 7.5 |
| C_{12/14} fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 Methacrylate structuring | 6 |
| Crosspolymer⁵⁾ | |
| Deionized Water | 27.4 |

### Example 11

### Unit Dose Formulation

A sufficient amount of exemplified microcapsules is weighed and mixed in a unit dose formulation to add the equivalent of 0.2% perfume.
The unit dose formulation can be contained in a PVOH (polyvinyl alcohol) film.

TABLE 11
Unit dose composition

| | Concentration |
|---|---|
| Ingredients | [wt %] |
| C12-C14 alkyl poly ethoxylate | 15 |
| C12-C14 alkyl poly ethoxylate sulfate Mono | 9.5 |
| Ethanol Amine salt | |
| Linear Alkylbenzene sulfonic acid | 17 |
| Citric Acid | 0.5 |
| C12-C18 Fatty Acid | 17 |
| Enzymes | 1.2 |
| Fluorescent brightener | 0.3 |
| 1,2 propanediol | 12 |
| Glycerol | 9 |
| Sodium Hydroxide | 1 |
| Mono Ethanol Amine | 6 |
| PDMS | 2.5 |
| Potassium sulphite | 0.2 |
| water | 8.8 |
| Total | 100 |

### Example 12

### Powder Detergent Composition

A sufficient amount of exemplified microcapsules is weighed and mixed in a powder detergent composition to add the equivalent of 0.2% perfume.

TABLE 12
Powder detergent composition

| Ingredients | Part |
|---|---|
| Anionic (Linear Alkyl Benzene Sulphonates) | 20% |
| Nonionics (Alcohol Ethoxylates (5-9 ethylene oxide) | 6% |
| Builders (zeolites, sodium carbonate) | 25% |
| Silicates | 6% |
| Sodium Sulphate | 35% |
| Others (Enzymes, Polymers, Bleach) | 7.5% |
| Spray-dried granule powder A-E | 0.5% |

### Example 13

### Concentrated All Purpose Cleaner Composition

A sufficient amount of exemplified microcapsules is weighed and mixed in a concentrated all-purpose cleaner composition to add the equivalent of 0.2% perfume.

TABLE 13
concentrated all-purpose cleaner composition

| | Amount | |
|---|---|---|
| Ingredients | (% wt) | Function |
| Ethoxylated Alcohol (C9-C11, 8EO) ⁽¹⁾ | 20 | Non-ionic surfactant |
| Sodium Dodecyl Benzene Sulfonate ⁽²⁾ | 16 | Anionic surfactant |
| Sodium Cumene Sulfonate ⁽³⁾ | 8 | Hydrotrope |
| Methyl chloro isothiazolinone Methyl | 0.8% | preservative |
| isothiazolinone 3.3:1 ⁽⁴⁾ | | |
| Water | 55.9 | solvent |

⁽¹⁾ Neodol 91-8 ®; trademark and origin: Shell Chemical
⁽²⁾ Biosoft D-40 ®; trademark and origin: Stepan Company
⁽³⁾ Stepanate SCS ®; trademark and origin: Stepan Company
⁽⁴⁾ Kathon CG ®; trademark and origin: Dow Chemical Company

All ingredients are mixed together and then the mixture was diluted with water to 100%.

### Example 14

### Solid Scent Booster Composition

The following compositions are prepared.

TABLE 14
Salt-based solid scent booster compositions

| Ingredients | Part |
|---|---|
| Sodium chloride | 95 |
| Spray-dried granule powder A-E | 5 |

TABLE 15
Urea-based solid scent booster compositions

| Ingredients | Part |
|---|---|
| Urea (beads) | 94 |
| Spray-dried granule powder A-E | 8 |
| Bentonite | 3 |
| Perfume | 3 |

### Example 15

### Shampoo Composition

A sufficient amount of exemplified microcapsules is weighed and mixed in a shampoo composition to add the equivalent of 0.2% perfume.

TABLE 16
Shampoo composition

| | Ingredients | Concentration [wt %] |
|---|---|---|
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

Polyquaternium-10 is dispersed in water. The remaining ingredients of phase A are mixed separately by addition of one after the other while mixing well after each adjunction. Then this pre-mix is added to the Polyquaternium-10 dispersion and was mixed for 5 min. Then Phase B and the premixed Phase C (heat to melt Monomuls 90L-12 in Texapon NSO IS) are added.
The mixture is mixed well. Then, Phase 0 and Phase E are added while agitating. The pH was adjusted with citric acid solution till pH: 5.5-6.0.

### Example 16

### Shampoo Composition

A sufficient amount of exemplified microcapsules is weighed and mixed in a shampoo composition to add the equivalent of 0.2% perfume.

TABLE 17
Shampoo composition

| | | Concentration |
|---|---|---|
| | Ingredients | [wt %] |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (&) C12-13 Pareth-4 (&) C12-13 | 2.5 |
| | Pareth-23 (&) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone & | 0.1 |
| | Methylisothiazolinone ¹¹⁾ | |
| | D-Panthenol 75% ¹²⁾ | 0.1 |
| | Water deionized | 0.3 |
| F | Sodium Chloride 25% aqueous sol. | 0.6 |
| | TOTAL: | 100 |

¹⁾ EDETA B Powder, BASF
²⁾ Jaguar C14 S, Rhodia
³⁾ Ucare Polymer JR-400, Noveon
⁴⁾ Sulfetal LA B-E, Zschimmer & Schwarz
⁵⁾ Zetesol LA, Zschimmer & Schwarz
⁶⁾ Tego Betain F 50, Evonik
⁷⁾ Xiameter MEM-1691, Dow Corning
⁸⁾ Lanette 16, BASF
⁹⁾ Comperlan 100, Cognis
¹⁰⁾ Cutina AGS, Cognis
¹¹⁾ Kathon CG, Rohm & Haas
¹²⁾ D-Panthenol, Roche

A premix comprising Guar Hydroxypropyltrimonium Chloride and Polyquaternium-10 are added to water and Tetrasodium EDTA while mixing. When the mixture is homogeneous, NaOH is added. Then, Phase C ingredients are added and the mixture was heat to 75° C. Phase D ingredients are added and mixed till homogeneous. The heating is stopped and temperature of the mixture is decreased to RT. At 45° C., ingredients of Phase E while mixing final viscosity is adjusted with 25% NaCl solution and pH of 5.5-6 is adjusted with 10% NaOH solution.

### Example 17

### Rinse-Off Hair Composition

A sufficient amount of exemplified microcapsules is weighed and mixed in a rinse-off composition to add the equivalent of 0.2% perfume.

TABLE 18
rinse-off composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | TOTAL: | 100 |

¹⁾ Genamin KDMP, Clariant
²⁾ Tylose H10 Y G4, Shin Etsu
³⁾ Lanette O, BASF
⁴⁾ Arlacel 165, Croda
⁵⁾ Incroquat Behenyl TMS-50-PA- (MH), Croda
⁶⁾ Brij S20, Croda
⁷⁾ Xiameter MEM-949, Dow Corning
⁸⁾ Alfa Aesar

Ingredients of Phase A are mixed until an uniform mixture was obtained. Tylose is allowed to completely dissolve. Then the mixture is heated up to 70-75° C. Ingredients of Phase B are combined and melted at 70-75° C. Then ingredients of Phase B are added to Phase A with good agitation and the mixing is continued until cooled down to 60° C. Then, ingredients of Phase C are added while agitating and keeping mixing until the mixture cooled down to 40° C. The pH is adjusted with citric acid solution till pH: 3.5-4.0.

### Example 18

### Antiperspirant Spray Anhydrous Composition

A sufficient amount of exemplified microcapsules is weighed and mixed in an antiperspirant spray anhydrous composition to add the equivalent of 0.2% perfume.

TABLE 19
antiperspirant spray anhydrous composition

| Ingredient | Amount (wt %) |
|---|---|
| Cyclomethicone¹⁾ | 53.51 |
| Isopropyl miristate | 9.04 |
| Silica²⁾ | 1.03 |
| Quaternium-18-Hectorite³⁾ | 3.36 |
| Aluminium Chlorohydrate⁴⁾ | 33.06 |

¹⁾Dow Corning ® 345 Fluid; trademark and origin: Dow Corning
²⁾Aerosil ® 200; trademark and origin: Evonik
³⁾Bentone ® 38; trademark and origin: Elementis Specialities
⁴⁾Micro Dry Ultrafine; origin: Reheis

Using a high speed stirrer, Silica and Quaternium-18-Hectorite are added to the Isopropyl miristate and Cyclomethicone mixture. Once completely swollen, Aluminium Chlorohydrate is added portion wise under stirring until the mixture was homogeneous and without lumps. The aerosol cans are filled with 25% Suspension of the suspension and 75% of Propane/Butane (2.5 bar).

### Example 19

### Antiperspirant Spray Emulsion Composition

A sufficient amount of exemplified microcapsules is weighed and mixed in antiperspirant spray emulsion composition to add the equivalent of 0.2% perfume.

TABLE 20
antiperspirant spray emulsion composition

| Ingredient | Amount (wt %) |
|---|---|
| Polysorbate 65¹⁾ (Part A) | 0.95 |
| Polyglyceryl-2 dipolyhydroxystearate²⁾ | 1.05 |
| (Part A) | |
| Cetyl PEG/PPG-10/1 Dimethicone³⁾ | 2.75 |
| (Part A) | |
| Cyclomethicone⁴⁾ (Part A) | 16.4 |
| Isopropylisostearate⁵⁾ (Part A) | 4.5 |
| Phenoxyethanol⁶⁾ (Part A) | 0.5 |
| Ethylhexylglycerin⁷⁾ (Part A) | 0.2 |
| C12-15 Alkyl Benzoate⁸⁾ (Part A) | 5.65 |
| Silica Silylate⁹⁾ (Part A) | 0.1 |
| Sodium Methylparaben¹⁰⁾ (Part B) | 0.1 |
| Aluminium Chlorohydrate¹¹⁾ (Part B) | 20 |
| Water (Part B) | 44.47 |
| Fragrance (Part C) | 3.33 |

¹⁾Tween 65; trademark and origin: CRODA
²⁾Dehymuls PGPH; trademark and origin: BASF
³⁾Abil EM-90; trademark and origin: BASF
⁴⁾Dow Corning 345 fluid; trademark and origin: Dow Corning
⁵⁾Crodamol ipis; trademark and origin: CRODA
⁶⁾Phenoxyethanol; trademark and origin: LANXESS
⁷⁾Sensiva sc 50; trademark and origin: KRAFT
⁸⁾Tegosoft TN; trademark and origin: Evonik
⁹⁾Aerosil R 812; trademark and origin: Evonik
¹⁰⁾Nipagin mna; trademark and origin: CLARIANT
¹¹⁾Locron L; trademark and origin: CLARIANT

The ingredients of Part A and Part B are weighted separately. Ingredients of Part A are heated up to 60° C. and ingredients of Part B are heated to 55° C. Ingredients of Part B are poured small parts while continuous stirring into A. Mixture were stirred well until the room temperature was reached. Then, ingredients of part C are added. The emulsion is mixed and is introduced into the aerosol cans. The propellant is crimped and added.
Aerosol filling: 30% Emulsion: 70% Propane/Butane 2.5 bar

### Example 20

### Deodorant Spray Composition

A sufficient amount of exemplified microcapsules is weighed and mixed in antiperspirant deodorant spray composition to add the equivalent of 0.2% perfume.

TABLE 21
deodorant spray composition

| Ingredient | Amount (wt %) |
|---|---|
| Ethanol 95% | 90.65 |
| Triclosan¹⁾ | 0.26 |
| Isopropyl miristate | 9.09 |

¹⁾Irgasan ® DP 300; trademark and origin: BASF

All the ingredients according to the sequence of the Table 24 are mixed and dissolved. Then the aerosol cans are filled, crimp and the propellant is added (Aerosol filling: 40% active solution 60% Propane/Butane 2.5 bar).

### Example 21

### Antiperspirant Roll-On Emulsion Composition

A sufficient amount of exemplified microcapsules is weighed and mixed in antiperspirant roll-on emulsion composition to add the equivalent of 0.2% perfume.

TABLE 22
antiperspirant roll-on emulsion composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to part B under stirring and the mixture is homogenized for 10 minutes. Then, the mixture is cooled down under stirring; and part C is slowly added when the mixture reached 45° C. and part D when the mixture reached at 35° C. while stirring. Then the mixture is cooled down to RT.

### Example 22

### Antiperspirant Roll-On Composition

A sufficient amount of exemplified microcapsules is weighed and mixed in antiperspirant roll-on composition to add the equivalent of 0.2% perfume.

TABLE 23
antiperspirant roll-on composition

| Ingredient | QUANTITY |
|---|---|
| Water (Part A) | 45 |
| Aluminum Chlorohydrate 50% aqueous solution¹⁾ (Part B) | 20 |
| Alcohol Denat. (Ethanol 96%) (Part B) | 30 |
| Ceteareth-12²⁾ (Part C) | 2 |
| Ceteareth-30³⁾ (Part C) | 2 |
| Fragrance (Part D) | 1 |

¹⁾LOCRON L; origin: CLARIANT
²⁾EUMULGIN B-1; origin: BASF
³⁾EUMULGIN B-3; origin: BASF

The ingredients of part B are mixed in the vessel then ingredient of part A is added. Then dissolved part C in part A and B. With perfume, 1 part of Cremophor RH40 for 1 part of perfume is added while mixing well

### Example 23

### Antiperspirant Roll-On Composition

A sufficient amount of exemplified microcapsules is weighed and mixed in antiperspirant roll-on emulsion composition to add the equivalent of 0.2% perfume.

TABLE 24
antiperspirant roll-on emulsion composition

| Ingredient | Amount (wt %) |
|---|---|
| Water (Part A) | 50.51 |
| Hydroxyethylcellulose¹⁾ (Part A) | 0.71 |
| Ethanol 95% (Part B) | 40.40 |
| 1,2-Propylene Glycol (Part B) | 5.05 |
| Triclosan²⁾ (Part B) | 0.30 |
| PEG-40 Hydrogenated castor oil³⁾ (Part C) | 3.03 |

¹⁾Natrosol ® 250 H; trademark and origin: Ashland
²⁾Irgasan ® DP 300; trademark and origin: BASF
³⁾Cremophor ® RH 40; trademark and origin: BASF

Part A is prepared by sprinkling little by little the Hydroxyethylcellulose in the water whilst rapidly stirring with the turbine. Stirring is continued until the Hydroxyethylcellulose is entirely swollen and giving a limpid gel. Then, Part B is poured little by little in Part A whilst continuing stirring until the whole is homogeneous. Part C is added.

### Example 24

### Deodorant Pump Without Alcohol Formulation

A sufficient amount of exemplified microcapsules is weighed and mixed in the following composition to add the equivalent of 0.2% perfume.

TABLE 25
deodorant composition

| Ingredients | Amount (wt %) |
|---|---|
| C12-15 Alkyl Lactate¹⁾ | 5 |
| Dimethicone²⁾ | 91.6 |
| Cetyl Lactate³⁾ | 1 |
| Octyldodecanol⁴⁾ | 0.8 |
| Triclosan⁵⁾ | 0.1 |
| PERFUME | 1.5 |

¹⁾Ceraphyl 41; trademark and origin ASHLAND
²⁾DOW CORNING 200 FLUID 0.65 cs; trademark and origin DOW CORNING CORPORATION
³⁾Ceraphyl 28; trademark and origin ASHLAND
⁴⁾Eutanol G; trademark and origin BASF
⁵⁾Irgasan ® DP 300; trademark and origin: BASF

All the ingredients are mixed according to the sequence of the table and the mixture is heated slightly to dissolve the Cetyl Lactate.

### Example 25

### Deodorant Pump with Alcohol Formulation

A sufficient amount of exemplified microcapsules is weighed and mixed in the following composition to add the equivalent of 0.2% perfume.

TABLE 26
deodorant composition

| Ingredients | Amount (wt %) |
|---|---|
| Ethyl Alcohol (Part A) | 60 |
| PEG-6 Caprylic/Capric Glycerides¹⁾ (Part A) | 2 |
| Water (Part A) | 35.6 |
| PEG-40 Hydrogenated Castor Oil²⁾ (Part B) | 0.4 |
| PERFUME (Part B) | 2 |

¹⁾Softigen 767; trademark and origin CRODA
²⁾Cremophor ® RH 40; trademark and origin: BASF

Ingredients from Part B are mixed together. Ingredients of Part A are dissolved according to the sequence of the Table and are poured into part B.

### Example 26

### Talc Formulation

A sufficient amount of granules A-E is weighed and mixed in introduced in a standard talc base: 100% talc, very slight characteristic odor, white powder, origin: LUZENAC to add the equivalent of 0.2% perfume.

### Example 27

### Shower-Gel Reference

A sufficient amount of exemplified microcapsules is weighed and mixed in the following composition to add the equivalent of 0.2% perfume.

TABLE 27
shower gel composition

| | Amount | |
|---|---|---|
| Ingredients | (% wt) | Function |
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% aqueous solution | 1.000 | pH adjuster |
| Cocamidopropyl Betaine⁴⁾ | 8.000 | Surfactant |
| Methylchloroisothiazolinone and | 0.100 | Preservative |
| Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

Ingredients are mixed, pH is adjusted to 6-6.3 (Viscosity: 4500 cPo+/−1500 cPo (Brookfield RV/Spindle #4/20 RPM)).

### Example 28

### Shower-Gel Composition

A sufficient amount of exemplified microcapsules is weighed and mixed in the following composition to add the equivalent of 0.2% perfume.

TABLE 28
shower gel composition

| | Amount | |
|---|---|---|
| Ingredients | (% wt) | Function |
| WATER deionized | 52.40 | Solvent |
| Tetrasodium EDTA ¹⁾ | 0.10 | Chelating agent |
| Sodium Benzoate | 0.50 | Preservative |
| Propylene Glycol | 2.00 | Solvent |
| Sodium C12-C15 Pareth Sulfate ²⁾ | 35.00 | Surfactant |
| Cocamidopropyl Betaine³⁾ | 8.00 | Surfactant |
| Polyquaternium-7⁴⁾ | 0.20 | Conditioning agent |
| Citric Acid (40%) | 1.00 | pH adjuster |
| Sodium Chloride | 0.80 | Viscosity adjuster |

¹⁾ EDETA B POWDER; trademark and origin: BASF
²⁾ ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

Ingredients are mixed, pH is adjusted to 4.5 (Viscosity: 3000 cPo+/−1500 cPo (Brookfield RV/Spindle #4/20 RPM)).

### Example 29

### Shower-Gel Composition

A sufficient amount of exemplified microcapsules is weighed and mixed in the following composition to add the equivalent of 0.2% perfume.

TABLE 29
shower gel composition

| | Amount | |
|---|---|---|
| Ingredients | (% wt) | Function |
| WATER deionized | 50.950 | Solvent |
| Tetrasodium EDTA ¹⁾ | 0.050 | Chelating agent |
| Sodium Benzoate | 0.500 | Preservative |
| Glycerin 86% | 3.500 | Solvent |
| Sodium Laureth Sulfate ²⁾ | 27.000 | Surfactant |
| Polyquaternium-7³⁾ | 1.000 | Conditioning Agent |
| Coco-Betaine⁴⁾ | 6.000 | Surfactant |
| PEG-120 Methyl Glucose trioleate⁵⁾ | 1.000 | Thickener |
| Citric Acid (40%) | 1.000 | pH adjuster |
| Glycol Distearate & Laureth-4 & | 3.000 | Pearlizing agent |
| Cocamidopropyl Betaine⁶⁾ | | |
| Sodium Chloride 20% | 5.000 | Viscosity adjuster |
| PEG-40 Hydrogenated Castor Oil⁷⁾ | 1.000 | Viscosity adjuster |

¹⁾ EDETA B POWDER; trademark and origin: BASF
²⁾ Texapon NSO IS; trademark and origin: COGNIS
³⁾MERQUAT 550; trademark and origin: LUBRIZOL
⁴⁾DEHYTON AB-30; trademark and origin: COGNIS
⁵⁾GLUCAMATE LT; trademark and origin: LUBRIZOL
⁶⁾EUPERLAN PK 3000 AM; trademark and origin: COGNIS
⁷⁾CREMOPHOR RH 40; trademark and origin: BASF

Ingredients are mixed, pH is adjusted to 4.5 (Viscosity: 4000 cPo+/−1500 cPo (Brookfield RV/Spindle #4/20 RPM))

### Example 30

### Hand Dishwash

A sufficient amount of exemplified microcapsules is weighed and mixed in the following composition to add the equivalent of 0.2% perfume.

TABLE 30
Hand dishwash composition

| | Amount | |
|---|---|---|
| Ingredients | (% wt) | Function |
| Linear alkylbenzene sulfonic acid ⁽¹⁾ | 20 | Anionic surfactant |
| Diethanolamide ⁽²⁾ | 3.5 | Foam booster |
| Sodium Hydroxide (50%) ⁽³⁾ | 3.4 | pH Adjuster/neutralizer |
| Secondary alcohol ethoxolate ⁽⁴⁾ | 2.5 | Non-ionic surfactant |
| Sodium xylene sulfonate | 6.3 | Hydrotrope |
| Water | 64.3 | Solvent |

⁽¹⁾ Biosoft S-118 ®; trademark and origin: Stepan Company
⁽²⁾ Ninol 40-CO ®; trademark and origin: Stepan Company
⁽³⁾ Stepanate SXS ®; trademark and origin: Stepan Company
⁽⁴⁾ Tergitol 15-S-9 ®; trademark and origin: Dow Chemical Company

Water with sodium hydroxide and diethanolamide are mixed. LAS is added. After the LAS is neutralized, the remaining ingredients are added. The pH was Checked (=7-8) and adjusted if necessary.

### Example 31

### Soap Bar Formulation

A soap bar composition including exemplified microcapsules is prepared at a concentration of 7.5% w/w.

TABLE 31
composition of soap formulation

| Ingredients | Soap Formulation (%) |
|---|---|
| Surfactants | |
| Sodium Linear Alkyl Benzene Solfonate | |
| Soap | 10 to 25 |
| Alpha Olefin Sulfonate (AOS) | |
| sodium lauryl sulfate | |
| Builders | |
| Sodium Carbonate | 5 to 15 |
| Sodium Tri Polyphospate | |
| Zeolite | |
| Fillers | |
| Sodium Sulphate | 5 to 30 |
| Talc | 5 to 30 |
| Dolamite | 5 to 50 |
| China Clay | 5 to 50 |
| Calcite - Calcium Carbonate | |
| Sodium Cholride | 5 to 20 |
| Structuring Ingredients | |
| Aluminium Sulphate | 0.5 to 5 |
| Alkaline Silicate | 1 to 5 |
| Others | |
| Colour | 0.1 to 1 |
| Perfume | 0.1 to 1 |
| Moisture | 5 to 15 |

### Example 32

### Toothpaste Formulation

A sufficient amount of a microcapsule slurry M (prepared according to the protocol disclosed in example 1 except that a menthol flavor is encapsulated) is weighed and mixed in the following composition to add the equivalent of 0.2% flavor.

TABLE 32
Toothpaste formulation

| Ingredients | Amount (% wt) |
|---|---|
| Polyethylene glycol 400 | 2.0% |
| Xanthan Gum | 0.6% |
| Sorbitol 70% Solution | 50% |
| Sodium Fluoride | 0.220% |
| Sodium Benzoate | 0.2% |
| Water | 15.230% |
| Hydrated Silica¹⁾ | 22.0% |
| Hydrated Silica²⁾ | 7.0% |
| Titanium Dioxide CI77891 | 0.5% |
| Sodium Lauryl Sulfate | 1.250% |
| Flavor | 1.2% |
| TOTAL | 100% |

¹⁾Tixosil 73
²⁾Tixosil 43

### Example 33

### Dicalcium Phosphate Based Toothpaste Formulation

A sufficient amount of a microcapsule slurry M (prepared according to the protocol disclosed in example 1 except that a menthol flavor is encapsulated) is weighed and mixed in the following composition to add the equivalent of 0.2% flavor.

TABLE 33
Toothpaste formulation

| Ingredients | Amount (% wt) |
|---|---|
| Sodium carboxymethyl cellulose | 1.2% |
| Flavor | 1.2% |
| DI/Purified Water | Q.S to Final Wt. |
| Sodium Lauryl Sulfate | 1.3% |
| Glycerine | 20.0% |
| Sodium Saccharin | 0.2% |
| Dicalcium phosphate dihydrate | 36.0% |
| Methylparaben | 0.2% |
| Silica¹⁾ | 3.0% |
| TOTAL | 100% |

¹⁾Aerosil ®200

### Example 34

### Mouthwash Alcohol Free Formulation

A sufficient amount of a microcapsule slurry M (prepared according to the protocol disclosed in example 1 except that a menthol flavor is encapsulated) is weighed and mixed in the following composition to add the equivalent of 0.2% flavor.

TABLE 34
Mouthwash formulation

| Ingredients | Amount (% wt) |
|---|---|
| Propylene Glycol | 10% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

### Example 35

### Mouthwash Formulation

A sufficient amount of a microcapsule slurry M (prepared according to the protocol disclosed in example 1 except that a menthol flavor is encapsulated) is weighed and mixed in the following composition to add the equivalent of 0.2% flavor.

TABLE 35
Mouthwash formulation

| Ingredients | Amount (% wt) |
|---|---|
| Ethyl Alcohol 190 Proof | 15.0% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.100% |
| Sucralose | 0.020% |
| Benzoic Acid | 0.050% |
| TOTAL | 100% |

1. A core-shell microcapsule comprising:
a core comprising a hydrophobic material, and
a polymeric shell comprising:
a polymeric material, and
a coacervate comprising a first polyelectrolyte and a second polyelectrolyte, wherein the first polyelectrolyte comprises a plant-protein.

2. The core-shell microcapsule according to claim 1, wherein the polymeric material and the coacervate are homogenously distributed within the shell.

3. The core-shell microcapsule according to claim 1, wherein the plant-protein is selected from the group consisting of potato protein, chickpea protein, pea protein, faba bean protein, barley protein, oat protein, soy protein, algae protein, wheat gluten protein, lupin protein, canola protein, hemp protein, rice protein, sunflower seed protein, and mixtures thereof.

4. The core-shell microcapsule according to claim 3, wherein the plant-protein is potato protein.

5. The core-shell microcapsule according to claim 1, wherein the second polyelectrolyte is selected from the group consisting of gum arabic, alginate salts, cellulose derivatives, guar gum, pectinate salts, pectin, carrageenan, polyacrylic and methacrylic acid, cellulose derivatives, xanthan gum, microbial exopolysaccharide, and mixtures thereof.

6. The core-shell microcapsule according to claim 5, wherein the second polyelectrolyte is gum arabic.

7. The core-shell microcapsule according to claim 1, wherein the polymeric material is selected from the group consisting of polyurea, polyester, polyurethane, polyamide, polyacrylate, polysiloxane, polycarbonate, polysulfonamide, poly (beta aminoester), polylactic acid, poly (thiol-acrylate), polymers of urea and formaldehyde, melamine and formaldehyde, melamine and urea, or melamine and glyoxal and mixtures thereof.

8. The core-shell microcapsule according to claim 1, wherein it comprises:
a core comprising a perfume oil, and
a shell comprising a polyurea and a coacervate comprising potato protein and gum arabic.

9. The core-shell microcapsule according to claim 1, wherein the polymeric material is present in an amount less than 10% by weight based on the total weight of the microcapsule.

10. A process for preparing a core-shell microcapsule comprising the steps of:
(i) providing a hydrophobic phase comprising a hydrophobic material and at least a polyfunctional monomer;
(ii) mixing a first polyelectrolyte and second polyelectrolyte in a dispersing phase under conditions sufficient to not form a suspension of complex coacervate; wherein the first polyelectrolyte comprises a plant-protein;
(iii) adding the hydrophobic phase into the dispersing phase to form a two phases dispersion and applying sufficient condition to form a coacervate; and
(iv) providing conditions sufficient to induce interfacial polymerization to form a core-shell microcapsule slurry.

11. A consumer product comprising:
a consumer product base; and
the core-shell microcapsule according to claim 1.

12. A fabric softener composition comprising:
a fabric softener active base;
the core-shell microcapsule according to claim 1; and
optionally free perfume oil.

13. A liquid detergent composition comprising:
a liquid detergent active base;
the core-shell microcapsule according to claim 1; and
optionally free perfume oil.

14. A solid detergent composition comprising:
a solid detergent active base;
the core-shell microcapsule according to claim 1; and
optionally free perfume oil.

15. The consumer product according to claim 11, wherein the consumer product is in the form of a home-care product or a personal care product.

16. The consumer product according to claim 12, wherein the fabric softener active base comprises at least one active material selected from the group consisting of dialkyl quaternary ammonium salts, dialkyl ester quaternary ammonium salts (esterquats), 1,2-stearoyl-3-trimethylammonium-propane (chloride salt), TEAQ (triethanolamine quat), silicones, and mixtures thereof.

17. The consumer product according to claim 16, wherein the active material is in an amount between 85% and 99.95% by weight based on the total weight of the composition.

18. The consumer product according to claim 13, wherein the liquid detergent active base comprises at least one active material selected from the group consisting of anionic surfactant, alkylbenzenesulfonate (ABS), secondary alkyl sulfonate (SAS), primary alcohol sulfate (PAS), lauryl ether sulfate (LES), methyl ester sulfonate (MES), nonionic surfactant, alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, and alkyl polyglucosamides.

19. The consumer product according to claim 18, wherein the active material is in an amount between 85% and 99.95% by weight based on the total weight of the composition.

20. The consumer product according to claim 14, wherein the solid detergent active base comprises at least one active material selected from the group consisting of anionic surfactant, alkylbenzenesulfonate (ABS), secondary alkyl sulfonate (SAS), primary alcohol sulfate (PAS), lauryl ether sulfate (LES), methyl ester sulfonate (MES), nonionic surfactant, alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, and alkyl polyglucosamides.
